(19) United States
(12) Patent Application Publication
(10) Pub. No.: US 2023/0243825 A1
(43) Pub. Date: Aug. 3, 2023

(54) ADAPTIVE NANOPARTICLE PLATFORMS FOR HIGH THROUGHPUT EXPANSION AND DETECTION OF ANTIGEN-SPECIFIC T CELLS

(71) Applicant: The Johns Hopkins University, Baltimore, MD (US)

(72) Inventors: Jonathan Schneck, Baltimore, MD (US); Hai-Quan Mao, Baltimore, MD (US); John Wirthlin Hickey, Baltimore, MD (US); Ariel Isser, Baltimore, MD (US)

(21) Appl. No.: 18/011,070

(22) PCT Filed: Jun. 23, 2021
(86) PCT No.: PCT/US2021/038676

§ 371 Date: Dec. 16, 2022

Related U.S. Application Data
(60) Provisional application No. 63/044,724, filed on Jun. 26, 2020.

(51) Int. Cl. G01N 33/569; G01N 33/543; C12N 5/0783

(52) U.S. Cl. CPC G01N 33/56972; G01N 33/54326; C12N 5/0636; G01N 2333/705

(57) ABSTRACT

Disclosed are methods for enriching and expanding antigen-specific T cells with paramagnetic nanoparticles comprising a major histocompatibility complex (MHC) or human leukocyte antigen (HLA) and a costimulatory molecule bound thereto. The platform eliminates the requirement of cell isolation and can be further adapted to be high throughput with the capability of processing multiple antigen-specific T cells in parallel. Accordingly, the disclosed methods provide a high-throughput workflow for the identification and analysis of antigen-specific T cell responses.

### FEDERALLY SPONSORED RESEARCH OR DEVELOPMENT

This invention was made with government support under CA108835, EB028239, EB023411, and CA229042 awarded by the National Institutes of Health. The government has certain rights in the invention.

### SEQUENCE LISTING

The text of the computer readable sequence listing filed herewith, titled “38583-601_SEQUENCE_LISTING_ST25”, created Jun. 23, 2021, having a file size of 2,392 bytes, is hereby incorporated by reference in its entirety.

### BACKGROUND

T cells are immune cells that play critical roles in carrying out and bolstering immune responses against pathogens, self, allergens, and cancer. Hickey et al., Biology of T Cells—Part A (2018). Each T cell recognizes antigenic peptide sequences presented in major histocompatibility complexes (MHC) through their unique T cell receptor (TCR). Identification of antigen-specific T cells is critical to understanding disease, Hickey et al., Biology of T Cells—Part A (2018); Singha et al., Nat. Nanotechnol. (2017); Tsai et al., Immunity (2010); Clemente-Casares et al., Nature (2016); Riddell et al., Nat. Med. (1996); and Walter et al., N. Engl. J. Med. (1995), and creating therapies. Fesnak et al., Nat. Rev. Cancer (2016); Rapoport et al., Nat. Med. (2015).

Despite their known roles in immunity, identification of antigen-specific T cells can be challenging. Unique TCRs are generated through VDJ recombination with 10¹⁴ possible unique TCRs, Davis and Bjorkman, Nature (1988), and the frequency of any one specific clonotype is between 1 in 10⁴ to 10⁶ of T cells. Jenkins and Moon, J. Immunol. (2012); Rizzuto et al., J. Exp. Med. (2009). This diversity and frequency require conventional methods of cellular identification, like flow or mass cytometry, to be adapted. Newell and Davis, Nat. Biotechnol. (2014).

Several approaches have been used to increase detection sensitivity, such as multimerizing MHC, Altman et al., Science (1996), co-evaluation with inflammatory markers (e.g., cytokines), Han et al., Lab Chip (2010); Betts et al., J. Immunol. Methods (2003); Frentsch et al., Nat. Med. (2005); Newell et al., Immunity 2012, and magnetic enrichment by peptide-loaded MHC (pMHC). Perica et al., ACS Nano 2015; Day et al., Invest. 2003. These techniques, however, also suffer from low throughput. To improve throughput, researchers have developed UV-cleavable peptides, Rodenko et al, Nat. Protoc. (2006), combinatorial fluorescent labeling, Hadrup et al., Nat. Methods (2009); Newell et al., Nat. Methods (2009), and pMHC yeast displays. Gee et al., Cell (2018). The high degree of complexity of these procedures, however, renders them difficult to use.

### SUMMARY

In some aspects, the presently disclosed subject matter provides a method for preparing one more adaptive artificial antigen presenting cells (aAPCs), the method comprising: (a) conjugating to a surface of a paramagnetic particle a major histocompatibility complex (MHC) or a human leukocyte antigen (HLA) and a costimulatory ligand to form a conjugated paramagnetic particle, wherein: (i) the magnetic particle has a diameter ranging from about 100 nm to about 5000 nm; and (ii) the MHC or HLA is not loaded with a peptide prior to conjugating the MHC or HLA to the surface of the paramagnetic particle; and (b) incubating the conjugated paramagnetic particle with one or more peptides to load the conjugated MHC or HLA on the paramagnetic particle with the one or more peptides to form one or more adaptive aAPCs.

In other aspects, the presently disclosed subject matter provides an adaptive artificial antigen presenting cell (aAPC) comprising a paramagnetic particle having a major histocompatibility complex (MHC) or human leukocyte antigen (HLA) and a costimulatory ligand conjugated to a surface thereof, wherein: (a) the magnetic particle has a diameter ranging from about 100 nm to about 5000 nm; (b) is capable of loading one or more antigen peptides through binding with the MHC or HLA prior to contacting T cells.

In other aspects, the presently disclosed subject matter provides a method for preparing one or more adaptive detection beads, the method comprising: (a) conjugating to a surface of a paramagnetic particle a major histocompatibility complex (MHC) or human leukocyte antigen (HLA) to form a conjugated paramagnetic particle, wherein: (i) the magnetic particle has a diameter ranging from about 100 nm to about 5000 nm; (ii) the MHC or HLA is not loaded with a peptide prior to conjugating the MHC or HLA to the surface of the paramagnetic particle; and (iii) the magnetic particle is labeled with a reporting moiety; and (b) incubating the conjugated paramagnetic particle with one or more peptides to load the conjugated MHC or HLA on the paramagnetic particle with the one or more peptides to form one or more adaptive detection beads.

In other aspects, the presently disclosed subject matter provides an adaptive detection bead comprising a paramagnetic particle having a major histocompatibility complex (MHC) or human leukocyte antigen (HLA) conjugated to a surface thereof, wherein: (a) the magnetic particle has a diameter ranging from about 100 nm to about 5000 nm; (b) is capable of loading one or more antigen peptides through binding with the MHC or HLA prior to contacting T cells; and (c) the magnetic particle is labeled with a reporting moiety.

In yet other aspects, the presently disclosed subject matter provides a method for identifying, isolating, or detecting one or more antigen-specific T cells, the method comprising: (a) contacting a plurality of unpurified immune cells comprising one or more antigen-specific T cells with a plurality of adaptive aAPCs prepared by the methods described hereinabove and/or a plurality of adaptive detection beads prepared by the methods described hereinabove; (b) placing a magnetic field in proximity to the plurality of adaptive aAPCs and/or the plurality of adaptive detection beads to separate antigen-specific T cells associated with the plurality of adaptive aAPCs and/or the plurality of adaptive detection beads from cells not associated with the plurality of adaptive aAPCs and/or the plurality of adaptive detection beads; (c) recovering antigen-specific T cells associated with the plurality of adaptive aAPCs and/or the plurality of adaptive detection beads; and (d) expanding the recovered antigen-specific T cells in culture for a period of time to provide a composition comprising antigen-specific T cells.

In some aspects, the presently disclosed subject matter provides a method for treating a disease, disorder, or condition, the method comprising administering to a subject in need of treatment thereof a composition comprising one or more antigen-specific T cells prepared by the method described hereinabove.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, FIG. 1F, FIG. 1G, and FIG. 1H show boosting activation of antigen-specific CD8+ T cells with co-culture of non-CD8+ T cells in E+E. (FIG. 1A) Schematic of eliminating CD8+ T cell isolation from protocol for using artificial antigen-presenting cells (aAPCs) for enrichment and expansion of antigen-specific T cells which represents cost, time and technical advantages. (FIG. 1B) Representative flow plot of CD8+ T cells (from B6 mouse) 7 days post enrichment and expansion from CD8+ T cells vs. splenocytes. (FIG. 1C) Percent and (FIG. 1D) number of antigen-specific T cells resulting from aAPC enrichment and expansion of two different starting populations of cells (purified CD8+ T cells vs. splenocytes) on day 7 (error bars show s.e.m.; **p<0.01, n=6, Student's t-test, two-tailed). (FIG. 1E) Number of antigen-specific T cells resulting from aAPC enrichment and expansion of CD8+ T cells, splenocytes, or isolated CD8+ T cells added back to splenocytes post-enrichment (error bars show s.e.m.; **p<0.01, ***p<0.001). (FIG. 1F) Cellular composition of post-enrichment fractions on days 0 and 7 during the culture (error bars show s.e.m., n=3). (FIG. 1G) Number of antigen-specific T cells 7 days after aAPC enrichment and expansion following depletion of CD4+ cells from splenocytes compared to isolated CD8+ T cells (error bars show s.e.m.;*p<0.05, ***p<0.001, n=3-4, one-way ANOVA with Tukey's post test). (FIG. 1H) Number of antigen-specific T cells resulting from aAPC enrichment and expansion from two different starting populations of cells (CD8+ T cell purified, Pan T cell purified) on day 7 (error bars show s.e.m.; **p<0.01, n=6, Student's t-test, two-tailed);

FIG. 2A, FIG. 2B, FIG. 2C, and FIG. 2D demonstrate that the throughput of enrichment and expansion of antigen-specific CD8+ T cells can be increased by increasing simultaneous parallel processing. (FIG. 2A) Schematic illustrating limitations of current approach known in the art to enrich rare cells by magnetic columns with 50-nm to 100-nm magnetic particles and increasing throughput by adapting a 96-well plate magnet approach with 300-nm magnetic nanoparticles. (FIG. 2B) Efficient enrichment and expansion from a starting population of splenocytes on a plate magnet in comparison to the CD8+ T cell isolated starting population (error bars show s.e.m.; p<0.05, n=8, Student's t-test, two-tailed). (FIG. 2C FIG. 2D) Comparison of (FIG. 2C) percentages and (FIG. 2D) numbers of four different antigen-specific T cell populations and comparing having the aAPCs batched versus processing individually in parallel (error bars show s.e.m.; n=7-14);

FIG. 3A, FIG. 3B, FIG. 3C, FIG. 3D, FIG. 3E, FIG. 3F, and FIG. 3G demonstrate increasing the throughput of E+E of antigen-specific CD8+ T cells through development of adaptive aAPCs. (FIG. 3A) Schematic illustrating limitations of current approach known in the art where individualized antigen-specific aAPCs require individual processing. It illustrates the concept of increasing throughput by creating adaptive aAPCs and then loading antigens post-conjugation and using a magnetic field for parallel processing. (FIG. 3B) CFSE dye dilution demonstrates effective antigen-specific activation of adaptive aAPCs compared to pre-loaded aAPCs. (FIG. 3C) IFN-γ secretions demonstrate functional responses after re-stimulating activated T cells with adaptive aAPCs compared to pre-loaded aAPCs (error bars show s.e.m., n=3, ***p<0.001, ****p<0.0001, One-way ANOVA with Tukey's Post-test). (FIG. 3D—FIG. 3E) Comparing the (FIG. 3D) fold enrichment and (FIG. 3E) percent cell recovery from adaptive aAPCs−peptide, adaptive aAPCs+peptide, and pre-loaded aAPCs in a doped Thy1.1+ system (error bars show s.e.m., n=8). (FIG. 3F—FIG. 3G) Cognate and non-cognate staining of antigen-specific cells enriched and expanded for 7 days by adaptive aAPCs compared to pre-loaded aAPCs (error bars show s.e.m., p>0.05, n=8-10);

FIG. 4A, FIG. 4B, FIG. 4C, and FIG. 4D demonstrate increasing the throughput of E+E of antigen-specific CD8+ T cells by parallel production of different detection beads. (FIG. 4A) Schematic illustrating limitations of current approaches known in the art which require creating individualized detection dimers/tetramers. It also illustrates how the throughput was increased by developing adaptive detection beads, which are loaded with peptide post-conjugation for parallel processing. (FIG. 4B) Adaptive detection beads are at least as sensitive as current detection technology for antigen-specific T cells at low, intermediate, and high frequencies at day 7 of the E+E protocol. (FIG. 4C) Percent and (FIG. 4D) number of antigen-specific CD8+ T cells enriched and expanded by peptide-loaded adaptive aAPCs and detected on day 7 by adaptive detection beads (n=3);

FIG. 5A, FIG. 5B, FIG. 5C, FIG. 5D, and FIG. 5E demonstrate application of the presently disclosed adaptive nanoparticle platforms to isolate and identify neoantigen-specific and human antigen-specific CD8+ T cells. (FIG. 5A) Schematic of in vivo vaccination protocol for both SIY and VDW peptides. (FIG. 5B) Representative flow plots and (FIG. 5C) summary of results from staining splenocytes of vaccinated B6 mice with SIY and VDW-loaded dimer and adaptive detection beads (error bars represent s.e.m., n=5, *p<0.05, ***p<0.001, ****p<0.0001, one-way ANOVA with Tukey's post test). (FIG. 5D) Replicates of staining of CMV, M-1 and MART-1 specific CD8+ T cells for antigen-specific (top panel) frequency and (bottom panel) number on Day 0 pre-enrichment and Days 7, 14, and 21 (for MART-1) following E+E (error bars represent s.e.m., n=1-5). (FIG. 5E) Representative staining of endogenous CMV-specific CD8+ T cells with adaptive detection beads compared to tetramer stains;

FIG. 6A, FIG. 6B, FIG. 6C, FIG. 6D, FIG. 6E, FIG. 6F, and FIG. 6G show data from enriching and expanding rare antigen-specific T cell populations directly from splenocytes and comparing to starting from purified CD8+ T cell populations. (FIG. 6A) Schematic of experimental set up for comparing different starting populations (splenocyte vs. purified CD8+ T cells). Harvested splenocytes were divided into two equal parts: one population that went through a step for CD8+ T cell isolation and the other that did not. Enriching from splenocytes does not alter antigen-specific (FIG. 6B) phenotype or (FIG. 6C) cytokine production on day 7 (error bars show s.e.m.; ns p>0.05, n=3, one-way ANOVA with Tukey's post test). (FIG. 6D-FIG. 6E) Enhancements in enrichment and expansion of antigen-specific CD8+ T cells from splenocyte starting populations do not come from increases in levels of fold enrichment or percent cell recovery of antigen-specific T cells on day 0. Doping fluorescently-labeled (CFSE) antigen-specific CD8+ T cells (2C or PMEL CD8+ T cells) at (1:10⁴) in endogenous splenocytes allow comparison of (FIG. 6D) fold enrichment and (FIG. 6E) percent cell recovery of 50 nm aAPCs that are not different from fold enrichment and cell recovery in CD8+ T cell populations (error bars show s.e.m.; n=5). (FIG. 6F) Percent and (FIG. 6G) number of antigen-specific T cells (TRP2+) resulting from aAPC enrichment and expansion two different starting populations of cells (CD8+ T cell purified, splenocytes) at day 7 (error bars show s.e.m.; *p<0.05, n=4, Student's t-test, two-tailed);

FIG. 7A and FIG. 7B demonstrate the importance of anti-CD28 for enrichment and expansion of CD8+ T cells from splenocytes. (FIG. 7A) Percentage and (FIG. 7B) number of KbSIY specific CD8+ T expanded with KbSIY+anti-CD28 vs. KbSIY-only aAPCs (error bars represent s.e.m.; *p<0.05, n=3-4, Student's t-test, two-tailed);

FIG. 8A and FIG. 8B demonstrate the contribution of endogenous antigen presenting cells to enhanced output from Splenocyte E+E. (FIG. 8A) Percent and (FIG. 8B) Number of antigen-specific T cells resulting from aAPC enrichment and expansion of splenocytes depleted of B cells (CD19+) macrophages (F4/80+) and dendritic cells (CD11c+) (error bars show s.e.m.; *p<0.05, n=3-4, one-way ANOVA);

FIG. 9A and FIG. 9B demonstrate understanding the contribution of CD4+ T cells in enhancing antigen-specific CD8+ T cell activation (FIG. 9A) Comparison staining of populations of splenocytes, Pan T cells, CD8+ isolation, and CD4+ depletion used for E+E experiments. (FIG. 9B) Percent of antigen-specific T cells resulting from aAPC enrichment and expansion from two different starting populations of cells (CD8+ T cell purified, Pan T cell purified) on day 7 (error bars show s.e.m.; **p<0.01, n=4, Student's t-test, two-tailed);

FIG. 10A, FIG. 10B, FIG. 10C, and FIG. 10D demonstrate establishing the proper dose of 300 nm aAPCs to use to enrich antigen-specific T cells. (FIG. 10A-FIG. 10B) Doping antigen-specific CD8+ T cells at (1:10⁴) in endogenous splenocytes allow comparison of (FIG. 10A) fold enrichment and (FIG. 10B) percent cell recovery of 300-nm aAPCs at different ratios of aAPCs to cells (error bars show s.e.m.; *p<0.05, **p<0.01, ***p<0.001, n=5, one-way ANOVA with Tukey's post test). (FIG. 10C) By doping in Thy1.1+, transgenic PMEL CD8+ T cells into Thy1.2+ mice at a 1:1000 ratio, we determine effective aAPC:Cell ratios needed for the new enrichment and expansion protocol (error bars show s.e.m.; *p<0.05, n=5, one-way ANOVA with Tukey's post test). (FIG. 10D) Comparing antigen-specific T cell frequency on day 7 from a 96-well plate format starting population of splenocytes or purified CD8+ T cells (error bars show s.e.m.; **p<0.01, n=8, Student's t-test, two-tailed);

FIG. 11 shows a schematic for comparing experimental set-up for comparing batched to individual antigen-specific CD8+ T cell enrichment and expansions;

FIG. 12A and FIG. 12B show the titration of detection bead:cell ratios to evaluate optimal staining concentration for staining antigen-specific T cells on day 7 of the enrichment and expansion protocol with a low final percentage of antigen-specific T cells. (FIG. 12A) Flow cytometry plots of peptide-loaded Adaptive aAPCs (Adaptive+Peptides) and unloaded (Adaptive−Peptides) detection beads (FIG. 12B) Percentage of control staining (Adaptive−Peptide/non-cognate) were subtracted to evaluate final percentage of antigen-specific T cells on day 7 and compare to traditional biotinylated dimer staining reagents;

FIG. 13A and FIG. 13B show the titration of detection bead:cell ratios to evaluate optimal staining concentration for staining antigen-specific T cells on day 7 of the enrichment and expansion protocol with an intermediate final percentage of antigen-specific T cells. (FIG. 13A) Flow cytometry plots of both peptide-loaded Adaptive aAPCs (Adaptive+Peptides) and unloaded aAPCs (Adaptive−Peptides) detection beads (FIG. 13B) Percentage of control staining (Adaptive−Peptide/non-cognate) were subtracted to evaluate final percentage of antigen-specific T cells on day 7 and compare to traditional biotinylated dimer staining reagents;

FIG. 14A and FIG. 14B show the titration of detection bead:cell ratios to evaluate optimal staining concentration for staining antigen-specific T cells on day 7 of the enrichment and expansion protocol with a high final percentage of antigen-specific T cells. (FIG. 14A) Flow cytometry plots of both peptide-loaded Adaptive aAPCs (Adaptive+Peptides) and unloaded (Adaptive−Peptides) detection beads (FIG. 14B) Percentage of control staining (Adaptive−Peptide/non-cognate) were subtracted to evaluate final percentage of antigen-specific T cells on day 7 and compare to traditional biotinylated dimer staining reagents;

FIG. 15 shows the combination of multiplexed Adaptive aAPC, 96-well plate enrichment and expansion starting from a population of splenocytes, and detection by Adaptive detection beads. Representative staining on day 7 by detection bead of antigen-specific T cells after enrichment and expansion for each antigen using unloaded adaptive detection beads (Adaptive-Peptide) as a negative control;

FIG. 16 shows a Peptide Stabilization Assay to Determine Relative Binding Affinity of SIY and VDW for Kb MHC molecule. RMA-S cells have significantly more Kb protein stabilized and expressed on their surface when pulsed with 1 μg of high affinity SIY peptide, compared to no peptide, 1 μg of db-restricted MCMV peptide, or 1 μg neoantigen VDW peptide (error bars show s.e.m; ****p<0.0001, n=3, one-way ANOVA with Tukey's post test);

FIG. 17A and FIG. 17B show In Vivo Peptide vaccination with VDW and SIY peptide. (FIG. 17A) Representative and (FIG. 17B) summary of non-cognate OVA and cognate SIY and VDW dimer staining of spleens and lymph nodes of unvaccinated and vaccinated mice. The frequency of SIY and VDW specific CD8+ T cells is significantly increased by D15 in the spleens of vaccinated mice (error bars show s.e.m; *p<0.05, **p<0.01, ****p<0.0001, n=5, one-way ANOVA with Tukey's post test);

FIG. 18A and FIG. 18B show Enrichment and Expansion of antigen-specific CD8+ T cells for viral (M-1 and CMV) and tumor (MART-1) antigens using Adaptive aAPCs. (FIG. 18A) Representative expansion data with 100 nm Adaptive aAPCs. (FIG. 18B) Representative expansion data with 300-nm Adaptive aAPCs.

FIG. 19 shows tumor-derived peptides can be eluted from the surface of tumor cells and then passively loaded onto adaptive aAPCs to activate tumor-specific T cells;

FIGS. 20A, FIG. 20B, FIG. 20C, and FIG. 20D demonstrate that the presently disclosed approach elutes B16-SIY below limit of detection by RMAS assay but detectable by Mass Spectrometry;

FIG. 21A, FIG. 21B, and FIG. 21C show that SIY peptide eluted from the surface of B16-SIY and pulsed onto adaptive aAPCs leads to robust 2C proliferation; and

FIG. 22A, FIG. 22B, FIG. 22C, FIG. 22D, FIG. 22E, and FIG. 22F, show that IFN-γ pre-treatment of B16-OVA tumor cells increases amount of peptide recovered and in-turn ability of eluate-pulsed adaptive aAPCs to stimulate OT-I T cells.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Adaptive Nanoparticle Platforms for High Throughput Expansion and Detection of Antigen-Specific T Cells

In some embodiments, the presently disclosed subject matter provides a method for preparing one more adaptive artificial antigen presenting cells (aAPCs), the method comprising: (a) conjugating to a surface of a paramagnetic particle a major histocompatibility complex (MHC) or human leukocyte antigen (HLA) and a costimulatory ligand to form a conjugated paramagnetic particle, wherein: (i) the magnetic particle has a diameter ranging from about 100 nm to about 5000 nm; and (ii) the MHC or HLA is not loaded with a peptide prior to conjugating the MHC or HLA to the surface of the paramagnetic particle; and (b) incubating the conjugated paramagnetic particle with one or more peptides to load the conjugated MHC or HLA on the paramagnetic particle with the one or more peptides to form one or more adaptive aAPCs.

In some embodiments, the major histocompatibility complex (MHC) is selected from the group consisting of an MHC-class I complex and an MHC-class II complex. In particular embodiments, the MHC-class I complex or MHC-class II complex comprises an MHC-Ig dimer (pMHC).

In certain embodiments, the costimulatory ligand is selected from the group consisting of an antibody or antigen-binding fragment thereof that specifically binds to CD28, CD80 (B7-1), CD86 (B7-2), B7-H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB. In particular embodiments, the costimulatory ligand comprises an antibody or antigen-binding fragment thereof that specifically binds to CD28.

In some embodiments, the one or more peptides can be the same or different and can be loaded individually or simultaneously from a mixture of 10,000 or more distinct antigens. In certain embodiments, the one or more peptides are eluted and/or isolated from a tumor cell or other cell expressing an antigen of interest. In particular embodiments, each distinct antigen is loaded onto the conjugated paramagnetic particle in a separate well of a multi-well microtiter plate. In some embodiments, the method further comprises washing the adaptive aAPCs magnetically.

The presently disclosed subject matter further comprises an adaptive aAPC prepared by the presently disclosed methods.

In other embodiments, the presently disclosed subject matter provides an adaptive artificial antigen presenting cell (aAPC) comprising a paramagnetic particle having a major histocompatibility complex (MHC) or human leukocyte antigen (HLA) and a costimulatory ligand conjugated to a surface thereof, wherein: (a) the magnetic particle has a diameter ranging from about 100 nm to about 5000 nm; (b) is capable of loading one or more antigen peptides through binding with the MHC or HLA prior to contacting T cells.

In certain embodiments, the major histocompatibility complex (MHC) is selected from the group consisting of an MHC-class I complex and an MHC-class II complex. In particular embodiments, the MHC-class I complex or the MHC-class II complex comprises an MHC-Ig dimer (pMHC).

In certain embodiments, the costimulatory ligand is selected from the group consisting of an antibody or antigen-binding fragment thereof that specifically binds to CD28, CD80 (B7-1), CD86 (B7-2), B7-H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB. In particular embodiments, the costimulatory ligand is selected from the group consisting of an antibody or antigen-binding fragment thereof that specifically binds to CD28.

In some embodiments, the one or more peptides can be the same or different and can be loaded individually or simultaneously from a mixture of 10,000 or more distinct antigens. In certain embodiments, the one or more peptides are eluted and/or isolated from a tumor cell or other cell expressing an antigen of interest.

In other embodiments, the presently disclosed subject matter provides a method for preparing one or more adaptive detection beads, the method comprising: (a) conjugating to a surface of a paramagnetic particle a major histocompatibility complex (MHC) or human leukocyte antigen (HLA) to form a conjugated paramagnetic particle, wherein: (i) the magnetic particle has a diameter ranging from about 100 nm to about 5000 nm; (ii) the MHC or HLA is not loaded with a peptide prior to conjugating the MHC or HLA to the surface of the paramagnetic particle; and (iii) the magnetic particle is labeled with a reporting moiety; and (b) incubating the conjugated paramagnetic particle with one or more peptides to load the conjugated MHC or HLA on the paramagnetic particle with the one or more peptides to form one or more adaptive detection beads.

In certain embodiments, the major histocompatibility complex (MHC) is selected from the group consisting of an MHC-class I complex and an MHC-class II complex. In particular embodiments, the MHC-class I complex or MHC-class II complex comprises an MHC-Ig dimer.

In certain embodiments, the one or more peptides can be the same or different and can be loaded individually or simultaneously from a mixture of 10,000 or more distinct antigens. In particular embodiments, the one or more peptides are eluted and/or isolated from a tumor cell or other cell expressing an antigen of interest. In some embodiments, each distinct antigen is loaded onto the conjugated paramagnetic particle in a separate well of a multi-well microtiter plate. In certain embodiments, the method further comprises washing the adaptive detection bead magnetically.

In particular embodiments, the reporting moiety comprises a fluorescent agent.

In some embodiments, the presently disclosed subject matter provides an adaptive detection bead prepared by the presently disclosed methods.

In other embodiments, the presently disclosed subject matter provides an adaptive detection bead comprising a paramagnetic particle having a major histocompatibility complex (MHC) or human leukocyte antigen (HLA) conjugated to a surface thereof, wherein: (a) the magnetic particle has a diameter ranging from about 100 nm to about 5000 nm; (b) is capable of loading one or more antigen peptides through binding with the MHC or HLA prior to contacting T cells; and (c) the magnetic particle is labeled with a reporting moiety.

In certain embodiments, the major histocompatibility complex (MHC) is selected from the group consisting of an MHC-class I complex and an MHC-class II complex. In particular embodiments, the MHC-class I complex or MHC-class II complex comprises an MHC-Ig dimer.

In some embodiments, the one or more peptides can be the same or different and can be loaded individually or simultaneously from a mixture of 10,000 or more distinct antigens. In particular embodiments, the one or more peptides are eluted and/or isolated from a tumor cell or other cell expressing an antigen of interest.

In particular embodiments, the reporting moiety comprises a fluorescent agent.

In yet other embodiments, the presently disclosed subject matter provides a method for identifying, isolating, or detecting one or more antigen-specific T cells, the method comprising: (a) contacting a plurality of unpurified immune cells comprising one or more antigen-specific T cells with a plurality of adaptive aAPCs prepared by the methods described hereinabove and/or a plurality of adaptive detection beads prepared by the methods described hereinabove; (b) placing a magnetic field in proximity to the plurality of adaptive aAPCs and/or the plurality of adaptive detection beads to separate antigen-specific T cells associated with the plurality of adaptive aAPCs and/or the plurality of adaptive detection beads from cells not associated with the plurality of adaptive aAPCs and/or the plurality of adaptive detection beads; (c) recovering antigen-specific T cells associated with the plurality of adaptive aAPCs and/or the plurality of adaptive detection beads; and (d) expanding the recovered antigen-specific T cells in culture for a period of time to provide a composition comprising antigen-specific T cells.

In certain embodiments, the plurality of unpurified immune cells comprising one or more antigen-specific T cells are obtained from a sample comprising one or more of a peripheral blood mononuclear cell (PBMC) sample, memory T cells, naive T cells, previously activated T cells, and tumor infiltrating lymphocytes. In particular embodiments, the plurality of unpurified immune cells comprising one or more antigen-specific T cells are obtained from a sample comprising one or more of bone marrow, lymph node tissue, spleen tissue, and a tumor. In more particular embodiments, the plurality of unpurified immune cells are obtained from a patient or a donor. In yet more particular embodiments, the donor comprises a donor who is HLA-matched to an adoptive transfer recipient. In certain embodiments, the plurality of unpurified immune cells are obtained from a patient and the patient has one or more diseases, disorders, or conditions selected from the group consisting of a cancer, an infectious disease, and an autoimmune disease.

In some embodiments, the one or more antigen-specific T cells are selected from the group consisting of cytotoxic T lymphocytes, helper T cells, and regulatory T cells. In particular embodiments, the one or more antigen-specific T cells are selected from the group consisting of CD8+ cytotoxic T lymphocytes, CD4+ helper T cells, and combinations thereof.

In some embodiments, the magnetic field comprises a magnetic field associated with a permanent magnet. In particular embodiments, the magnetic field comprises a magnetic field associated with a neodymium magnet.

In some embodiments, the expanding of the recovered cells in culture for a period of time is performed on a multi-well microtiter plate. In certain embodiments, the multi-well microtiter plate comprises a 96-well microtiter plate.

In some embodiments, a purity of the expanded recovered antigen-specific T cells is improved relative to a method in which the antigen-specific T cells are isolated from the plurality of unpurified immune cells prior to contacting the plurality of unpurified immune cells with the plurality of paramagnetic nanoparticles.

In other embodiments, a percent of antigen-specific T cells is increased relative to a method in which the antigen-specific T cells are isolated from the plurality of unpurified immune cells prior to contacting the plurality of unpurified immune cells with the plurality of paramagnetic nanoparticles.

In yet other embodiments, a number of antigen-specific T cells is increased relative to a method in which the antigen-specific T cells are isolated from the plurality of unpurified immune cells prior to contacting the plurality of unpurified immune cells with the plurality of paramagnetic nanoparticles.

In some embodiments, the presently disclosed subject matter provides a method for treating a disease, disorder, or condition, the method comprising administering to a subject in need of treatment thereof a composition comprising one or more antigen-specific T cells prepared by the method described hereinabove.

In certain embodiments, the disease, disorder, or condition is selected from the group consisting of a cancer, an infectious disease, and an autoimmune disease. In particular embodiments, the disease, disorder, or condition is a cancer and the one or more antigen-specific T cells comprise cytotoxic T cells specific for one or more tumor-associated peptide antigens to the subject in need of treatment thereof. In yet more particular embodiments, the cancer comprises a solid tumor or a hematological malignancy.

In certain embodiments, the cancer is selected from the group consisting of a melanoma, colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, nonsmall cell lung carcinoma, small-cell lung cancer, mesothelioma, transitional and squamous cell urinary carcinoma, brain cancer, a neuroblastoma, and a glioma.

### A. Representative Sources of Immune Cells

As provided hereinabove, in some embodiments, the presently disclosed methods involve enrichment and expansion of antigen-specific T cells, including, but not limited to, cytotoxic T lymphocytes (CTLs), helper T cells, and regulatory T cells. In some embodiments, the presently disclosed methods involve enrichment and expansion of antigen-specific CTLs.

Precursor T cells can be obtained from a patient or from a suitable HLA-matched donor. Precursor T cells can be obtained from a number of sources, including, but not limited to, peripheral blood mononuclear cells (PBMC), bone marrow, lymph node tissue, spleen tissue, tumors, and combinations thereof. In some embodiments, the T cells are obtained from a PBMC sample from a patient. In some embodiments, the PBMC sample is used to isolate the T cell population of interest, such as CD8+, CD4+ or regulatory T cells. In some embodiments, precursor T cells are obtained from a unit of blood collected from a patient or a donor using any number of techniques known to the skilled artisan, such as Ficoll separation. For example, precursor T cells from the circulating blood of a patient or a donor can be obtained by apheresis or leukapheresis. The apheresis product typically contains lymphocytes, including T cells and precursor T cells, monocytes, granulocytes, B cells, other nucleated white blood cells, red blood cells, and platelets. Leukapheresis is a laboratory procedure in which white blood cells are separated from a sample of blood.

Cells collected by apheresis can be washed to remove the plasma fraction and to place the cells in an appropriate buffer or media for subsequent processing steps. Washing steps can be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample can be removed, and the cells directly re-suspended in a culture medium.

If desired, precursor T cells can be isolated from peripheral blood lymphocytes by lysing the red blood cells and depleting the monocytes, for example, by centrifugation through a PERCOLL™ gradient. In certain embodiments, the sample from which the T cells are obtained can be used without any isolation or preparatory steps.

If desired, subpopulations of T cells can be separated from other cells that may be present. For example, specific subpopulations of T cells, such as CD28+, CD4+, CD8+, CD45RA+, and CD45RO+ T cells, can be further isolated by positive or negative selection techniques. Other enrichment techniques include cell sorting and/or selection via negative magnetic immunoadherence or flow cytometry, e.g., using a cocktail of monoclonal antibodies directed to cell surface markers present on the cells negatively selected.

In certain embodiments, leukocytes are collected by leukapheresis, and are subsequently enriched for CD8+ T cells using known processes, such as magnetic enrichment columns that are commercially available. The CD8-enriched cells are then enriched for antigen-specific T cells using magnetic enrichment with the aAPC reagent. In various embodiments, at least about 10⁵, or at least about 10⁶, or at least about 10⁷ CD8-enriched cells are isolated for antigen-specific T cell enrichment.

B. Artificial Antigen Presenting Cells (aAPCs) Comprising Magnetic Particles

As provided hereinabove, the sample comprising the immune cells (e.g., CD8+ T cells) is contacted with an artificial Antigen Presenting Cell (aAPC) comprising a particle having magnetic properties. In some embodiments, such particles are nanoparticles and are referred to herein as “nano-aAPCs.” Paramagnetic materials have a small, positive susceptibility to magnetic fields. These materials are attracted by a magnetic field and the material does not retain the magnetic properties when the external field is removed. Exemplary paramagnetic materials include, without limitation, magnesium, molybdenum, lithium, tantalum, and iron oxide. Paramagnetic beads suitable for magnetic enrichment are commercially available (e.g., DYNABEADS®, MACS MICROBEADS™, Miltenyi Biotec, and the like). In some embodiments, the aAPC particle comprises an iron dextran bead (e.g., a dextran-coated iron-oxide bead).

In certain embodiments, the aAPCs contain at least two ligands, an antigen presenting complex (e.g., a major histocompatibility complex (MHC), including a peptide-MHC), and a costimulatory ligand, e.g., a lymphocyte activating ligand. Antigen presenting complexes comprise an antigen binding cleft, which harbors an antigen for presentation to a T cell or T cell precursor. Antigen presenting complexes can be, for example, MHC class I or class II molecules, and can be linked or tethered to provide dimeric or multimeric MHC. In some embodiments, the MHC are monomeric, but their close association on the paramagnetic nanoparticle is sufficient for avidity and activation. In some embodiments, the MHC are dimeric. Dimeric MHC class I constructs can be constructed by fusion to immunoglobulin heavy chain sequences, which are then associated through one or more disulfide bonds (and with associated light chains). In some embodiments, the signal 1 complex is a non-classical MHC-like molecule, such as member of the CD1 family (e.g., CD1a, CD1b, CD1c, CD1d, and CD1e). MHC multimers can be created by direct tethering through peptide or chemical linkers, or can be multimeric via association with streptavidin through biotin moieties. In some embodiments, the antigen presenting complexes are MHC class I or MHC class II molecular complexes involving fusions with immunoglobulin sequences, which are extremely stable and easy to produce, based on the stability and secretion efficiency provided by the immunoglobulin backbone.

MHC class I molecular complexes having immunoglobulin sequences are described in U.S. Pat. No. 6,268,411, which is hereby incorporated by reference in its entirety. These MIIC class I molecular complexes may be formed in a conformationally intact fashion at the ends of immunoglobulin heavy chains. MHC class I molecular complexes to which antigenic peptides are bound can stably bind to antigen-specific lymphocyte receptors (e.g., T cell receptors). In various embodiments, the immunoglobulin heavy chain sequence is not full length, but comprises an Ig hinge region, and one or more of CH1, CH2, and/or CH3 domains. The Ig sequence may or may not comprise a variable region, but where variable region sequences are present, the variable region may be full or partial. The complex may further comprise immunoglobulin light chains.

Exemplary MIIC class I molecular complexes comprise at least two fusion proteins. A first fusion protein comprises a first MHC class I a chain and a first immunoglobulin heavy chain (or portion thereof comprising the hinge region), and a second fusion protein comprises a second MHC class I a chain and a second immunoglobulin heavy chain (or portion thereof comprising the hinge region). The first and second immunoglobulin heavy chains associate to form the MHC class I molecular complex, which comprises two MHC class I peptide-binding clefts. The immunoglobulin heavy chain can be the heavy chain of an IgM, IgD, IgG1, IgG3, IgG2β, IgG2α, IgG4, IgE, or IgA. In some embodiments, an IgG heavy chain is used to form MHC class I molecular complexes. If multivalent MHC class I molecular complexes are desired, IgM or IgA heavy chains can be used to provide pentavalent or tetravalent molecules, respectively.

Exemplary class I molecules include HLA-A, HLA-B, HLA-C, HLA-E, and these may be employed individually or in any combination. In some embodiments, the antigen presenting complex is an HLA-A2 ligand.

Exemplary MHC class II molecular complexes are described in U.S. Pat. Nos. 6,458,354, 6,015,884, 6,140,113, and 6,448,071, which are hereby incorporated by reference in their entireties. MHC class II molecular complexes comprise at least four fusion proteins. Two first fusion proteins comprise (i) an immunoglobulin heavy chain (or portion thereof comprising the hinge region) and (ii) an extracellular domain of an MHC class IIβ chain. Two second fusion proteins comprise (i) an immunoglobulin κ or λ light chain (or portion thereof) and (ii) an extracellular domain of an MHC class Ha chain. The two first and the two second fusion proteins associate to form the MHC class II molecular complex. The extracellular domain of the MIIC class IIβ chain of each first fusion protein and the extracellular domain of the MHC class IIα chain of each second fusion protein form an MHC class II peptide binding cleft.

The immunoglobulin heavy chain can be the heavy chain of an IgM, IgD, IgG3, IgG1, IgG2β, IgG2α, IgG4, IgE, or IgA. In some embodiments, an IgG1 heavy chain is used to form divalent molecular complexes comprising two antigen binding clefts. Optionally, a variable region of the heavy chain can be included. IgM or IgA heavy chains can be used to provide pentavalent or tetravalent molecular complexes, respectively.

Fusion proteins of an MIIC class II molecular complex can comprise a peptide linker inserted between an immunoglobulin chain and an extracellular domain of an MHC class II polypeptide. The length of the linker sequence can vary, depending upon the flexibility required to regulate the degree of antigen binding and receptor cross linking.

Immunoglobulin sequences in some embodiments are humanized monoclonal antibody sequences.”

The presently disclosed paramagnetic nano-aAPC also can have a costimulatory molecule bound thereto. Such costimulatory molecules can be referred to herein as a “Signal 2.” Such costimulatory molecules are generally a T cell affecting molecule, that is, a molecule that has a biological effect on a precursor T cell or on an antigen-specific T cell. Such biological effects include, for example, differentiation of a precursor T cell into a CTL, helper T cell (e.g., Th1, Th2), or regulatory T cell; and/or proliferation of T cells. Thus, T cell affecting molecules include T cell costimulatory molecules, adhesion molecules, T cell growth factors, and regulatory T cell inducer molecules. In some embodiments, an aAPC comprises at least one such ligand; optionally, an aAPC comprises at least two, three, or four such ligands.

In certain embodiments, signal 2 is a T cell costimulatory molecule. T cell costimulatory molecules contribute to the activation of antigen-specific T cells. Such molecules include, but are not limited to, molecules that specifically bind to CD28 (including antibodies), CD80 (B7-1), CD86 (B7-2), B7-H3, 4-1BB, 4-1BBL, CD27, CD30, CD134 (OX-40L), B7h (B7RP-1), CD40, LIGHT, antibodies that specifically bind to HVEM, antibodies that specifically bind to CD40L, antibodies that specifically bind to OX40, and antibodies that specifically bind to 4-1BB. In some embodiments, the costimulatory molecule (signal 2) is an antibody (e.g., a monoclonal antibody) or portion thereof, such as F(ab′)2, Fab, scFv, or single chain antibody, or other antigen binding fragment. In some embodiments, the antibody is a humanized monoclonal antibody or portion thereof having antigen-binding activity, or is a fully human antibody or portion thereof having antigen-binding activity. Adhesion molecules useful for nano-aAPC can be used to mediate adhesion of the nano-aAPC to a T cell or to a T cell precursor. Useful adhesion molecules include, for example, ICAM-1 and LFA-3.

In some embodiments, signal 1 is provided by peptide-HLA-A2 complexes, and signal 2 is provided by B7.1-Ig or anti-CD28. An exemplary anti-CD28 monoclonal antibody is 9.3 mAb (Tan et al., J. Exp. Med. 1993 177:165), which may be humanized in certain embodiments and/or conjugated to the bead as a fully intact antibody or an antigen-binding fragment thereof.

Some embodiments employ T cell growth factors, which affect proliferation and/or differentiation of T cells. Examples of T cell growth factors include cytokines (e.g., interleukins, interferons) and superantigens. If desired, cytokines can be present in molecular complexes comprising fusion proteins, or can be encapsulated by the aAPC. Particularly useful cytokines include IL-2, IL-4, IL-7, IL-10, IL-12, IL-15, IL-21 gamma interferon, and CXCL10. Optionally, cytokines are provided solely by media components during expansion steps.

The nanoparticles can be made of any material, and materials can be appropriately selected for the desired magnetic property, and may comprise, for example, metals such as iron, nickel, cobalt, or alloy of rare earth metal. Paramagnetic materials also include magnesium, molybdenum, lithium, tantalum, and iron oxide. Paramagnetic beads suitable for enrichment of materials (including cells) are commercially available, and include iron dextran beads, such as dextran-coated iron oxide beads. In embodiments of the presently disclosed subject matter where magnetic properties are not required, nanoparticles can also be made of nonmetal or organic (e.g., polymeric) materials such as cellulose, ceramics, glass, nylon, polystyrene, rubber, plastic, or latex. In exemplary material for preparation of nanoparticles is poly(lactic-co-glycolic acid) (PLGA) and copolymers thereof, which may be employed in connection with these embodiments. Other materials including polymers and co-polymers that may be employed include those described in PCT/US2014/25889, which is hereby incorporated by reference in its entirety.

In some embodiments, the magnetic particles are biocompatible. This characteristic is particularly important in embodiments where the aAPC will be delivered to the patient in association with the enriched and expanded cells. For example, in some embodiments, the magnetic particles are biocompatible iron dextran paramagnetic beads.

In particular embodiments, the particle has a size (e.g., average diameter) of between about 100 nm to about 5000 nm, including about 100 nm, 200 nm, 300 nm, 400 nm, 500 nm, 600 nm, 700 nm, 800 nm, 900 nm, 1000 nm, 2000 nm, 3000 nm, 4000 nm, and 5000 nm. In some embodiments, the particle has a size of between about 100 nm to about 500 nm, including about 100 nm, 150 nm, 200 nm, 250 nm, 300 nm, 350 nm, 400 nm, 450 nm, and 500 nm. In particular embodiments, the particle has a size of about 300 nm. This size of magnetic nanoparticle affords the ability to use less expensive, lower power magnets, such as neodymium magnets associated with multi-well plates, to separate antigen-specific T cells associated with the magnetic nanoparticles. In previous embodiments, for example those disclosed in U.S. Pat. No. 10,098,939, which is incorporated herein by reference in its entirety, smaller superparamagnetic nanoparticles, e.g., 20 nm to about 200 nm, were used. These superparamagnetic nanoparticles of a smaller size required high gradient magnetic fields generated by specialized magnetic particle columns required to amplify the magnetic field strength.

Receptor-ligand interactions at the cell-nanoparticle interface are not well understood. Nanoparticle binding and cellular activation, however, are sensitive to membrane spatial organization, which is particularly important during T cell activation, and magnetic fields can be used to manipulate cluster-bound nanoparticles to enhance activation. See WO/2014/150132. For example, T cell activation induces a state of persistently enhanced nanoscale TCR clustering and nanoparticles are sensitive to this clustering in a way that larger particles are not. See WO/2014/150132, which is incorporated herein by reference in its entirety.

Furthermore, nanoparticle interactions with TCR clusters can be exploited to enhance receptor triggering. T cell activation is mediated by aggregation of signaling proteins, with “signaling clusters” hundreds of nanometers across, initially forming at the periphery of the T cell-APC contact site and migrating inward. As described herein, an external magnetic field can be used to enrich antigen-specific T cells (including rare naive cells) and to drive aggregation of magnetic nano-aAPC bound to TCR, resulting in aggregation of TCR clusters and enhanced activation of naive T cells. Magnetic fields can exert appropriately strong forces on paramagnetic particles, but are otherwise biologically inert, making them a powerful tool to control particle behavior. T cells bound to paramagnetic nano-aAPC are activated in the presence of an externally applied magnetic field. Nano-aAPC are themselves magnetized, and attracted to both the field source and to nearby nanoparticles in the field, inducing bead and thus TCR aggregation to boost aAPC-mediated activation. See WO/2014/150132.

Nano-aAPCs bind more TCR on and induce greater activation of previously activated compared to naive T cells. In addition, application of an external magnetic field induces nano-aAPC aggregation on naive cells, enhancing T cells proliferation both in vitro and following adoptive transfer in vivo. Importantly, in a melanoma adoptive immunotherapy model, T cells activated by nano-aAPC in a magnetic field mediate tumor rejection. Thus, the use of applied magnetic fields permits activation of naive T cell populations, which otherwise are poorly responsive to stimulation. This is an important feature of immunotherapy as naive T cells have been shown to be more effective than more differentiated subtypes for cancer immunotherapy, with higher proliferative capacity and greater ability to generate strong, long-term T cell responses. Thus, nano-aAPC can used for magnetic field enhanced activation of T cells to increase the yield and activity of antigen-specific T cells expanded from naive precursors, improving cellular therapy for example, patients with infectious diseases, cancer, or autoimmune diseases, or to provide prophylactic protection to immunosuppressed patients.

Molecules can be directly attached to nanoparticles by adsorption or by direct chemical bonding, including covalent bonding. See, Hermanson, BIOCONJUGATE TECHNIQUES, Academic Press, New York, 1996. A molecule itself can be directly activated with a variety of chemical functionalities, including nucicophilic groups, leaving groups, or electrophilic groups. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, a molecule can be bound to a nanoparticle through the use of a small molecule-coupling reagent. Non-limiting examples of coupling reagents include carbodiimides, maleimides, n-hydroxysuccinimide esters, bischloroethylamines, bifunctional aldehydes such as glutaraldehyde, anyhydrides and the like. In other embodiments, a molecule can be coupled to a nanoparticle through affinity binding such as a biotin-streptavidin linkage or coupling, as is well known in the art. For example, streptavidin can be bound to a nanoparticle by covalent or non-covalent attachment, and a biotinylated molecule can be synthesized using methods that are well known in the art.

If covalent binding to a nanoparticle is contemplated, the support can be coated with a polymer that contains one or more chemical moieties or functional groups that are available for covalent attachment to a suitable reactant, typically through a linker. For example, amino acid polymers can have groups, such as the s-amino group of lysine, available to couple a molecule covalently via appropriate linkers. This disclosure also contemplates placing a second coating on a nanoparticle to provide for these functional groups.

Activation chemistries can be used to allow the specific, stable attachment of molecules to the surface of nanoparticles. There are numerous methods that can be used to attach proteins to functional groups. For example, the common cross-linker glutaraldehyde can be used to attach protein amine groups to an aminated nanoparticle surface in a two-step process. The resultant linkage is hydrolytically stable. Other methods include use of cross-linkers containing n-hydrosuccinimido (NHS) esters which react with amines on proteins, cross-linkers containing active halogens that react with amine-, sulfhydryl-, or histidine-containing proteins, cross-linkers containing epoxides that react with amines or sulfhydryl groups, conjugation between maleimide groups and sulfhydryl groups, and the formation of protein aldehyde groups by periodate oxidation of pendant sugar moieties followed by reductive amination.

The ratio of particular ligands on the same nanoparticle can be varied to increase the effectiveness of the nanoparticle in antigen or costimulatory ligand presentation. For example, nanoparticles can be coupled with IILA-A2-Ig and anti-CD28 at a variety of ratios, such as about 30:1, about 25:1, about 20:1, about 15:1, about 10:1, about 5:1, about 3:1, about 2:1, about 1:1, about 0.5:1, about 0.3:1; about 0.2:1, about 0.1:1, or about 0.03:1. The total amount of protein coupled to the supports may be, for example, about 250 mg/mL, about 200 mg/mL, about 150 mg/mL, about 100 mg/mL, or about 50 mg/mL of particles. Because effector functions such as cytokine release and growth may have differing requirements for Signal 1 versus Signal 2 than T cell activation and differentiation, these functions can be determined separately.

The configuration of nanoparticles can vary from being irregular in shape to being spherical and/or from having an uneven or irregular surface to having a smooth surface. Non-spherical aAPCs are described in WO 2013/086500, which is hereby incorporated by reference in its entirety.

The aAPCs present antigen to T cells and thus can be used to both enrich for and expand antigen-specific T cells, including from naive T cells. The peptide antigens will be selected based on the desired therapy, for example, cancer, type of cancer, infectious disease, and the like. In some embodiments, the method is conducted to treat a cancer patient, and neoantigens specific to the patient are identified, and synthesized for loading aAPCs. In some embodiments, between three and ten neoantigens are identified through genetic analysis of the tumor (e.g., nucleic acid sequencing), followed by predictive bioinformatics. As shown herein, several antigens can be employed together (on separate aAPCs), with no loss of functionality in the method. In some embodiments, the antigens are natural, non-mutated, cancer antigens, of which many are known. This process for identifying antigens on a personalized basis is described in greater detail below.

A variety of antigens can be bound to antigen presenting complexes. The nature of the antigens depends on the type of antigen presenting complex that is used. For example, peptide antigens can be bound to MHC class I and class II peptide binding clefts. Non-classical MHC-like molecules can be used to present non-peptide antigens such as phospholipids, complex carbohydrates, and the like (e.g., bacterial membrane components such as mycolic acid and lipoarabinomannan). Any peptide capable of inducing an immune response can be bound to an antigen presenting complex. Antigenic peptides include tumor-associated antigens, autoantigens, alloantigens, and antigens of infectious agents.

The terms “cancer-specific antigen (CSA)” and “tumor-specific antigen (TSA)” are used interchangeably herein and refer to a protein, carbohydrate, or other molecule that is uniquely expressed by and/or displayed on cancer cells and is not expressed by or displayed on other cells in the body (e.g., normal healthy cells). In contrast, the terms “cancer-associated-antigen (CAA)” and “tumor-associated-antigen (TAA)” are used interchangeably herein and refer to a protein, carbohydrate, or other molecule that is not uniquely expressed by or displayed on a tumor cell and instead also is expressed on normal cells under certain conditions. Cancer-specific antigens and cancer-associated antigens are well known in the art. In some embodiments, the CSA or CAA comprises one or more antigenic cancer epitopes associated with a malignant cancer or tumor, a metastatic cancer or tumor, or a leukemia. A cancer “neoantigen” is a novel cancer-specific antigen that arises as a consequence of tumor-specific mutations (T. N. Schumacher and R. D. Schreiber, Science, 348(6230):69-74 (2015); and T. C. Wirth and F. Kühnel, Front Immunol., 8: 1848 (2017)).

“Tumor-associated antigens” include unique tumor antigens expressed exclusively by the tumor from which they are derived, shared tumor antigens expressed in many tumors but not in normal adult tissues (oncofetal antigens), and tissue-specific antigens expressed also by the normal tissue from which the tumor arose. Tumor associated antigens can be, for example, embryonic antigens, antigens with abnormal post-translational modifications, differentiation antigens, products of mutated oncogenes or tumor suppressors, fusion proteins, or oncoviral proteins. A variety of tumor-associated antigens are known in the art, and many of these are commercially available. Oncofetal and embryonic antigens include carcinoembryonic antigen and alpha-fetoprotein (usually only highly expressed in developing embryos but frequently highly expressed by tumors of the liver and colon, respectively), MAGE-1 and MAGE-3 (expressed in melanoma, breast cancer, and glioma), placental alkaline phosphatase sialyl-Lewis X (expressed in adenocarcinoma), CA-125 and CA-19 (expressed in gastrointestinal, hepatic, and gynecological tumors), TAG-72 (expressed in colorectal tumors), epithelial glycoprotein 2 (expressed in many carcinomas), pancreatic oncofetal antigen, 5T4 (expressed in gastriccarcinoma), alphafetoprotein receptor (expressed in multiple tumor types, particularly mammary tumors), and M2A (expressed in germ cell neoplasia).

Tumor-associated differentiation antigens include tyrosinase (expressed in melanoma) and particular surface immunoglobulins (expressed in lymphomas). Mutated oncogene or tumor-suppressor gene products include Ras and p53, both of which are expressed in many tumor types, Her-2/neu (expressed in breast and gynecological cancers), EGF-R, estrogen receptor, progesterone receptor, retinoblastoma gene product, myc (associated with lung cancer), ras, p53, nonmutant associated with breast tumors, MAGE-1, and MAGE-3 (associated with melanoma, lung, and other cancers). Fusion proteins include BCR-ABL, which is expressed in chromic myeloid leukemia. Oncoviral proteins include HPV type 16, E6, and E7, which are found in cervical carcinoma.

Tissue-specific antigens include melanotransferrin and MUC1 (expressed in pancreatic and breast cancers); CD10 (previously known as common acute lymphoblastic leukemia antigen, or CALLA) or surface immunoglobulin (expressed in B cell leukemias and lymphomas); the a chain of the IL-2 receptor, T cell receptor, CD45R, CD4+/CD8+ (expressed in T cell leukemias and lymphomas); prostate specific antigen and prostatic acid-phosphatase (expressed in prostate carcinoma); GP 100, MelanA/Mart-1, tyrosinase, gp75/brown, BAGE, and S-100 (expressed in melanoma); cytokeratins (expressed in various carcinomas); and CD19, CD20, and CD37 (expressed in lymphoma).

Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas).

“Antigens of infectious agents” include components of protozoa, bacteria, fungi (both unicellular and multicellular), viruses, prions, intracellular parasites, helminths, and other infectious agents that can induce an immune response. Bacterial antigens include antigens of gram-positive cocci, gram positive bacilli, gram-negative bacteria, anaerobic bacteria, such as organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaccae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma.

Antigens of protozoan infectious agents include antigens of malarial plasmodia, Leishmania species, Trypanosoma species and Schistosoma species. Fungal antigens include antigens of Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia.

Viral peptide antigens include, but are not limited to, those of adenovirus, herpes simplex virus, papilloma virus, respiratory syncytial virus, poxviruses, HIV, influenza viruses, and CMV. Particularly useful viral peptide antigens include HIV proteins such as HIV gag proteins (including, but not limited to, membrane anchoring (MA) protein, core capsid (CA) protein and nucleocapsid (NC) protein), HIV polymerase, influenza virus matrix (M) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like.

Antigens, including antigenic peptides, can be bound to an antigen binding cleft of an antigen presenting complex either actively or passively, as described in U.S. Pat. No. 6,268,411, which is hereby incorporated by reference in its entirety. Optionally, an antigenic peptide can be covalently bound to a peptide binding cleft.

If desired, a peptide tether can be used to link an antigenic peptide to a peptide binding cleft. For example, crystallographic analyses of multiple class I MHC molecules indicate that the amino terminus of (32M is very close, approximately 20.5 Angstroms away, from the carboxyl terminus of an antigenic peptide resident in the MHC peptide binding cleft. Thus, using a relatively short linker sequence, approximately 13 amino acids in length, one can tether a peptide to the amino terminus of (32M. If the sequence is appropriate, that peptide will bind to the MHC binding groove (see U.S. Pat. No. 6,268,411).

Antigen-specific T cells which are bound to the aAPCs can be separated from cells which are not bound using magnetic enrichment, or other cell sorting or capture technique. Other processes that can be used for this purpose include flow cytometry and other chromatographic means (e.g., involving immobilization of the antigen-presenting complex or other ligand described herein). In one embodiment antigen-specific T cells are isolated (or enriched) by incubation with beads, for example, antigen-presenting complex/anti-CD28-conjugated paramagnetic beads (such as DYNABEADS®), for a time period sufficient for positive selection of the desired antigen-specific T cells.

In some embodiments, a population of T cells can be substantially depleted of previously active T cells using, e.g., an antibody to CD44, leaving a population enriched for naive T cells. Binding nano-aAPCs to this population would not substantially activate the naive T cells, but would permit their purification.

In still other embodiments, ligands that target NK cells, NKT cells, or B cells (or other immune effector cells), can be incorporated into a paramagnetic nanoparticle, and used to magnetically enrich for these cell populations, optionally with expansion in culture as described below. Additional immune effector cell ligands are described in PCT/US2014/25889, which is hereby incorporated by reference in its entirety.

Without wishing to be bound by theory, removal of unwanted cells may reduce competition for cytokines and growth signals, remove suppressive cells, or may simply provide more physical space for expansion of the cells of interest.

Enriched T cells are then expanded in culture within the proximity of a magnet to produce a magnetic field, which enhances T cell receptor clustering of aAPC bound cells. Cultures can be stimulated for variable amounts of time (e.g., about 0.5, 2, 6, 12, 36, 48, or 72 hours as well as continuous stimulation) with nano-aAPC. The effect of stimulation time in highly enriched antigen-specific T cell cultures can be assessed. Antigen-specific T cell can be placed back in culture and analyzed for cell growth, proliferation rates, various effector functions, and the like, as is known in the art. Such conditions may vary depending on the antigen-specific T cell response desired. In some embodiments, T cells are expanded in culture from about 2 days to about 3 weeks, or in some embodiments, about 5 days to about 2 weeks, or about 5 days to about 10 days. In some embodiments, the T cells are expanded in culture for about 1 week, after which time a second enrichment and expansion step is optionally performed. In some embodiments, 2, 3, 4, or 5 enrichment and expansion rounds are performed.

After the one or more rounds of enrichment and expansion, the antigen-specific T cell component of the sample will be at least about 1% of the cells, or in some embodiments, at least about 5%, at least about 10%, at least about 15%, or at least about 20%, or at least about 25% of the cells in the sample. Further, these T cells generally display an activated state. From the original sample isolated from the patient, the antigen-specific T cells in various embodiments are expanded from about 100-fold to about 10,000 fold, such as at least about 1000-fold, at least about 2000-fold, at least about 3,000 fold, at least about 4,000-fold, or at least about 5,000-fold in various embodiments. After the one or more rounds of enrichment and expansion, at least about 10⁶, or at least about 10⁷, or at least about 10⁸, or at least about 10⁹ antigen-specific T cells are obtained.

The effect of nano-aAPC on expansion, activation and differentiation of T cell precursors can be assayed in any number of ways known to those of skill in the art. A rapid determination of function can be achieved using a proliferation assay, by determining the increase of CTL, helper T cells, or regulatory T cells in a culture by detecting markers specific to each type of T cell. Such markers are known in the art. CTL can be detected by assaying for cytokine production or for cytolytic activity using chromium release assays.

In addition to generating antigen-specific T cells with appropriate effector functions, another parameter for antigen-specific T cell efficacy is expression of homing receptors that allow the T cells to traffic to sites of pathology (Sallusto et al., Nature 401, 708-12, 1999; Lanzavecchia & Sallusto, Science 290, 92-97, 2000). For example, effector CTL efficacy has been linked to the following phenotype of homing receptors, CD62L+, CD45RO+, and CCR7−. Thus, a nano-aAPC-induced and/or expanded CTL population can be characterized for expression of these homing receptors. Homing receptor expression is a complex trait linked to initial stimulation conditions. Presumably, this is controlled both by the costimulatory complexes as well as cytokine milieu. One important cytokine that has been implicated is IL-12 (Salio et al., 2001). As discussed below, nano-aAPC offer the potential to vary individually separate components (e.g., T cell effector molecules and antigen presenting complexes) to optimize biological outcome parameters. Optionally, cytokines such as IL-12 can be included in the initial induction cultures to affect honing receptor profiles in an antigen-specific T cell population.

Optionally, a cell population comprising antigen-specific T cells can continue to be incubated with either the same nano-aAPC or a second nano-aAPC for a period of time sufficient to form a second cell population comprising an increased number of antigen-specific T cells relative to the number of antigen-specific T cells in the first cell population. Typically, such incubations are carried out for 3-21 days, preferably 7-10 days.

Suitable incubation conditions (culture medium, temperature, etc.) include those used to culture T cells or T cell precursors, as well as those known in the art for inducing formation of antigen-specific T cells using DC or artificial antigen presenting cells. See, e.g., Latouche & Sadelain, Nature Biotechno. 18, 405-09, April 2000; Levine et al., J. Immunol. 159, 5921-30, 1997; Maus et al., Nature Biotechnol. 20, 143-48, February 2002. See also the specific examples, below.

To assess the magnitude of a proliferative signal, antigen-specific T cell populations can be labeled with CFSE and analyzed for the rate and number of cell divisions. T cells can be labeled with CFSE after one-two rounds of stimulation with nano-aAPC to which an antigen is bound. At that point, antigen-specific T cells should represent 2-10% of the total cell population. The antigen-specific T cells can be detected using antigen-specific staining so that the rate and number of divisions of antigen-specific T cells can be followed by CFSE loss. At varying times (for example, 12, 24, 36, 48, and 72 hours) after stimulation, the cells can be analyzed for both antigen presenting complex staining and CFSE. Stimulation with nano-aAPC to which an antigen has not been bound can be used to determine baseline levels of proliferation. Optionally, proliferation can be detected by monitoring incorporation of 3H-thymidine, as is known in the art.

### C. Methods for Personalized Medicine

In some embodiments, the presently disclosed subject matter provides methods for personalized medicine, including cancer immunotherapy. The methods are accomplished using the aAPCs to identify antigens to which the patient will respond, followed by administration of the appropriate peptide-loaded aAPC to the patient, or followed by enrichment and expansion of the antigen specific T cells ex vivo.

Genome-wide sequencing has dramatically altered our understanding of cancer biology. Sequencing of cancers has yielded important data regarding the molecular processes involved in the development of many human cancers. Driving mutations have been identified in key genes involved in pathways regulating three main cellular processes (1) cell fate, (2) cell survival and (3) genome maintenance. Vogelstein et al., Science 339, 1546-58 (2013).

Genome-wide sequencing also has the potential to revolutionize our approach to cancer immunotherapy. Sequencing data can provide information about both shared as well as personalized targets for cancer immunotherapy. In principle, mutant proteins are foreign to the immune system and are putative tumor-specific antigens. Indeed, sequencing efforts have defined hundred if not thousands of potentially relevant immune targets. Limited studies have shown that T cell responses against these neo-epitopes can be found in cancer patients or induced by cancer vaccines. However, the frequency of such responses against a particular cancer and the extent to which such responses are shared between patients are not well known. One of the main reasons for our limited understanding of tumor-specific immune responses is that current approaches for validating potential immunologically relevant targets are cumbersome and time consuming.

Thus, in some embodiments, the presently disclosed subject matter provides a high-throughput platform-based approach for detection of T cell responses against neo-antigens in cancer. This approach uses the aAPC platform described herein for the detection of even low-frequency T cell responses against cancer antigens. Understanding the frequency and between-person variability of such responses would have important implications for the design of cancer vaccines and personalized cancer immunotherapy.

Although central tolerance abrogates T cell responses against self-proteins, oncogenic mutations induce neo-epitopes against which T cell responses can form. Mutation catalogues derived from whole exome sequencing provide a starting point for identifying such neo-epitopes. Using HLA binding prediction algorithms (Srivastava, PLoS One 4, e6094 (2009), it has been predicted that each cancer can have up 7-10 neo-epitopes. A similar approach estimated hundreds of tumor neo-epitopes. Such algorithms, however, may have low accuracy in predicting T cell responses, and only 10% of predicted HLA-binding epitopes are expected to bind in the context of HLA (Lundegaard C, Immunology 130, 309-18 (2010)). Thus, predicted epitopes must be validated for the existence of T cell responses against those potential neo-epitopes.

In certain embodiments, the nano-aAPC system is used to screen for neo-epitopes that induce a T cell response in a variety of cancers, or in a particular patient's cancer. Cancers may be genetically analyzed, for example, by whole exome-sequencing. For example, of a panel of 24 advanced adenocarcinomas, an average of about 50 mutations per tumor were identified. Of approximately 20,000 genes analyzed, 1327 had at least one mutation, and 148 had two or more mutations. 974 missense mutations were identified, with a small additional number of deletions and insertions.

A list of candidate peptides can be generated from overlapping nine amino acid windows in mutated proteins. All nine-AA windows that contain a mutated amino acid, and 2 non-mutated “controls” from each protein will be selected. These candidate peptides will be assessed computationally for MHC binding using a consensus of MHC binding prediction algorithms, including NetMHC and stabilized matrix method (SMM). Nano-aAPC and MHC binding algorithms have been developed primarily for HLA-A2 allele. The sensitivity cut-off of the consensus prediction can be adjusted until a tractable number of mutation containing peptides (approximately 500) and non-mutated control peptides (approximately 50) are identified.

A peptide library is then synthesized. MHC (e.g., A2) bearing aAPC are deposited in multi well plates and passively loaded with peptide. CD8 T cells may be isolated from PBMC of both A2 positive healthy donors and A2 positive pancreatic cancers patients (or other cancer or disease described herein). Subsequently, the isolated T cells are incubated with the loaded aAPCs in the plates for the enrichment step. Following the incubation, the plates are placed on a magnetic field and the supernatant containing irrelevant T cells not bound to the aAPCs is removed. The remaining T cells that are bound to the aAPCs will be cultured and allowed to expand for 7 to 21 days. Antigen specific expansion is assessed by re-stimulation with aAPC and intracellular IFNγ fluorescent staining.

In some embodiments, a patient's T cells are screened against an array or library of nanoAPCs, and the results are used for diagnostic or prognostic purposes. For example, the number and identity of T cell anti-tumor responses against mutated proteins, overexpressed proteins, and/or other tumor-associated antigens can be used as a biomarker to stratify risk. For example, the number of such T cell responses may be inversely proportionate to the risk of disease progression or risk of resistance or non-responsiveness to chemotherapy. In other embodiments, the patient's T cells are screened against an array or library of nano-APCs, and the presence of T cells responses, or the number or intensity of these T cells responses identifies that the patient has a sub-clinical tumor, and/or provides an initial understanding of the tumor biology.

In some embodiments, a patient or subject's T cells are screened against an array or library of paramagnetic aAPCs, each presenting a different candidate peptide antigen. This screen can provide a wealth of information concerning the subject or patient's T cell repertoire, and the results are useful for diagnostic or prognostic purposes. For example, the number and identity of T cell anti-tumor responses against mutated proteins, overexpressed proteins, and/or other tumor-associated antigens can be used as a biomarker to stratify risk, to monitor efficacy of immunotherapy, or predict outcome of immunotherapy treatment. Further, the number or intensity of such T cell responses may be inversely proportionate to the risk of disease progression or may be predictive of resistance or non-responsiveness to chemotherapy. In other embodiments, a subject's or patient's T cells are screened against an array or library of nano-APCs each presenting a candidate peptide antigen, and the presence of T cells responses, or the number or intensity of these T cells responses, provides information concerning the health of the patient, for example, by identifying autoimmune disease, or identifying that the patient has a sub-clinical tumor. In these embodiments, the process not only identifies a potential disease state, but provides an initial understanding of the disease biology.

### C.1 Methods for Treating a Disease, Disorder, or Condition

In some embodiments, the presently disclosed subject matter provides methods for treating a disease, disorder, or condition through immunotherapy in which detection, enrichment and/or expansion of antigen-specific immune cells ex vivo is therapeutically or diagnostically desirable. Accordingly, the presently disclosed subject matter is generally applicable for detecting, enriching and/or expanding antigen-specific T cells, including cytotoxic T lymphocytes (CTLs), helper T cells, and regulatory T cells.

Antigen-specific T cells obtained using nano-aAPC, can be administered to patients by any appropriate routes, including intravenous administration, intra-arterial administration, subcutaneous administration, intradermal administration, intralymphatic administration, and intratumoral administration. Patients include both human and veterinary patients.

Antigen-specific regulatory T cells can be used to achieve an immunosuppressive effect, for example, to treat or prevent graft versus host disease in transplant patients, or to treat or prevent autoimmune diseases, such as those listed above, or allergies. Uses of regulatory T cells are disclosed, for example, in US 2003/0049696, US 2002/0090724, US 2002/0090357, US 2002/0034500, and US 2003/0064067, which are hereby incorporated by reference in their entireties. Antigen-specific T cells prepared according to these methods can be administered to patients in doses ranging from about 5-10×10⁶ CTL/kg of body weight (approximately 7×10⁸ CTL/treatment) up to about 3.3×10⁹ CTL/kg of body weight (approximately 6×10⁹ CTL/treatment) (Walter et al., New England Journal of Medicine 333, 1038-44, 1995; Yee et al., J Exp Med 192, 1637-44, 2000). In other embodiments, patients can receive about 10³, about 5×10³, about 10⁴, about 5×10⁴, about 10⁵, about 5×10⁵, about 10⁶, about 5×10⁶, about 10⁷, about 5×10⁷, about 10⁸, about 5×10⁸, about 10⁹, about 5×10⁹, or about 10¹⁰ cells per dose administered intravenously. In still other embodiments, patients can receive intranodal injections of, e.g., about 8×10⁶ or about 12×10⁶ cells in a 200 μL bolus. Doses of nano-APC that are administered with cells include about 10³, about 5×10³, about 10⁴, about 5×10⁴, about 10⁵, about 5×10⁵, about 10⁶, about 5×10⁶, about 10⁷, about 5×10⁷, about 10⁸, about 5×10⁸, about 10⁹, about 5×10⁹, or about 10¹⁰ nano-aAPC per dose.

In an exemplary embodiment, the enrichment and expansion process is performed repeatedly on the same sample derived from a patient. A population of T cells is enriched and activated on Day 0, followed by a suitable period of time (e.g., about 3-20 days) in culture. Subsequently, nano-aAPC can be used to again enrich and expand against the antigen of interest, further increasing population purity and providing additional stimulus for further T cell expansion. The mixture of nano-aAPC and enriched T cells may subsequently again be cultured in vitro for an appropriate period of time, or immediately re-infused into a patient for further expansion and therapeutic effect in vivo. Enrichment and expansion can be repeated any number of times until the desired expansion is achieved.

In some embodiments, a cocktail of nano-aAPC, each against a different antigen, can be used at once to enrich and expand antigen T cells against multiple antigens simultaneously. In this embodiment, a number of different nano-aAPC batches, each bearing a different MHC-peptide, would be combined and used to simultaneously enrich T cells against each of the antigens of interest. The resulting T cell pool would be enriched and activated against each of these antigens, and responses against multiple antigens could thus be cultured simultaneously. These antigens could be related to a single therapeutic intervention; for example, multiple antigens present on a single tumor.

In some embodiments, the patient receives immunotherapy with one or more checkpoint inhibitors, prior to receiving the antigen-specific T cells by adoptive transfer, or prior to direct administration of aAPCs bearing neoantigens identified in vitro through genetic analysis of the patient's tumor. In various embodiments, the checkpoint inhibitor(s) target one or more of CTLA-4 or PD-1/PD-L1, which may include antibodies against such targets, such as monoclonal antibodies, or portions thereof, or humanized or fully human versions thereof. In some embodiments, the checkpoint inhibitor therapy comprises ipilimumab or Keytruda (pembrolizumab).

In some embodiments, the patient receives about 1 to 5 rounds of adoptive immunotherapy (e.g., one, two, three, four or five rounds). In some embodiments, each administration of adoptive immunotherapy is conducted simultaneously with, or after (e.g., from about 1 day to about 1 week after), a round of checkpoint inhibitor therapy. In some embodiments, adoptive immunotherapy is provided about 1 day, about 2 days, or about 3 days after checkpoint inhibitor therapy.

In still other embodiments, adoptive transfer or direct infusion of nano-aAPCs to the patient comprises, as a ligand on the bead, a ligand that targets one or more of CTLA-4 or PD-1/PD-L1. In these embodiments, the method can avoid certain side effects of administering soluble checkpoint inhibitor therapy.

### C.1.1 Methods for Treating Cancer

In some embodiments, the disease, disorder, or condition is a cancer. In particular embodiments, the cancer is a solid tumor or a hematological malignancy. The enrichment and expansion of antigen-specific CTLs ex vivo for adoptive transfer to a patient provides for a robust anti-tumor immune response.

Cancers that can be treated or evaluated according to the presently disclosed methods include cancers that historically illicit poor immune responses or have a high rate of recurrence. Exemplary cancers include various types of solid tumors, including carcinomas, sarcomas, and lymphomas. In various embodiments the cancer is melanoma (including metastatic melanoma), colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, neuroblastoma, and glioma. In some embodiments, the cancer is a hematological malignancy, such as chronic myelogenous leukemia, childhood acute leukemia, non-Hodgkin's lymphomas, chronic lymphocytic leukemia, malignant cutaneous T-cells, mycosis fungoids, non-MF cutaneous T-cell lymphoma, lymphomatoid papulosis, T-cell rich cutaneous lymphoid hyperplasia, and discoid lupus erythematosus.

In various embodiments, the cancer is stage I, stage II, stage III, or stage IV. In some embodiments, the cancer is metastatic and/or recurrent. In some embodiments, the cancer is preclinical, and is detected in the screening system described herein (e.g., colon cancer, pancreatic cancer, or other cancer that is difficult to detect early).

### C.1.2 Method for Treating an Infectious Disease

In other embodiments, the presently disclosed subject matter includes a method for treating an infectious disease. The infectious disease may be one in which enrichment and expansion of antigen-specific immune cells (such as CD8+ or CD4+ T cells) ex vivo for adoptive transfer to the patient could enhance or provide for a productive immune response. Infectious diseases that can be treated include those caused by bacteria, viruses, prions, fungi, parasites, helminths, and the like. Such diseases include AIDS, hepatitis, CMV infection, and post-transplant lymphoproliferative disorder (PTLD).

CMV, for example, is the most common viral pathogen found in organ transplant patients and is a major cause of morbidity and mortality in patients undergoing bone marrow or peripheral blood stem cell transplants. This is due to the immunocompromised status of these patients, which permits reactivation of latent virus in seropositive patients or opportunistic infection in seronegative individuals. A useful alternative to these treatments is a prophylactic immunotherapeutic regimen involving the generation of vims-specific CTL derived from the patient or from an appropriate donor before initiation of the transplant procedure. PTLD occurs in a significant fraction of transplant patients and results from Epstein-Barr virus (EBV) infection. EBV infection is believed to be present in approximately 90% of the adult population in the United States. Active viral replication and infection is kept in check by the immune system, but, as in cases of CMV, individuals immunocompromised by transplantation therapies lose the controlling T cell populations, which permits viral reactivation. This represents a serious impediment to transplant protocols. EBV may also be involved in tumor promotion in a variety of hematological and non-hematological cancers.

Other viral pathogens potentially treated by the presently disclosed methods include, but are not limited to adenovirus, herpes simplex virus, papilloma virus, respiratory syncytial virus, poxviruses, HIV, influenza viruses, and COVID-19.

### C.1.3 Method for Treating an Autoimmune Disease

In some embodiments, the patient has an autoimmune disease, in which enrichment and expansion of regulatory T cells (e.g., CD4+, CD25+, Foxp3+) ex vivo for adoptive transfer to the patient could dampen the deleterious immune response. Autoimmune diseases that can be treated include systemic lupus erythematosus, rheumatoid arthritis, type I diabetes, multiple sclerosis, Crohn's disease, ulcerative colitis, psoriasis, myasthenia gravis, Goodpasture's syndrome, Graves' disease, pemphigus vulgaris, Addison's disease, dermatitis herpetiformis, celiac disease, and Hashimoto's thyroiditis. In some embodiments, the patient is suspected of having an autoimmune disease or immune condition (such as those described in the preceding sentence), and the evaluation of T cell responses against a library of paramagnetic nano-aAPCs as described herein, is useful for identifying or confirming the immune condition.

### D. Reagents/Kits

In other embodiments, the presently disclosed subject matter provides a kit comprising the presently disclosed nano-aAPCs together with components for performing the enrichment and expansion process. Suitable containers for the presently disclosed paramagnetic nanoparticles include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). Optionally, one or more different antigens can be bound to the paramagnetic nanoparticles or can be supplied separately. Kits may comprise, alternatively or in addition, one or more multi-well plates or culture plates for T cells. In some embodiments, kits comprise a sealed container comprising paramagnetic nanoparticles, a magnet, and optionally test tubes and/or solution or buffers for performing magnetic enrichment.

A kit can further comprise a second container comprising a pharmaceutically acceptable buffer, such as phosphate-buffered saline, Ringer's solution, or dextrose solution. It can also contain other materials useful to an end user, including other buffers, diluents, filters, needles, and syringes.

Kits also may contain reagents for assessing the extent and efficacy of antigen-specific T cell activation or expansion, such as antibodies against specific marker proteins, MHC class I or class II molecular complexes, TCR molecular complexes, anticlonotypic antibodies, and the like.

A kit can also comprise a package insert containing written instructions for methods of inducing antigen-specific T cells, expanding antigen-specific T cells, using paramagnetic nanoparticles in the kit in various protocols. The package insert can be an unapproved draft package insert or can be a package insert approved by the Food and Drug Administration (FDA) or other regulatory body.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Adaptive Nanoparticle Platforms for High Throughput Expansion and Detection of Antigen-Specific T Cells

### 1.1 Overview

T cells are critical players in disease, yet their antigen-specificity has been difficult to identify as current techniques are limited in terms of sensitivity, throughput, or ease of use. To address these challenges, the presently disclosed subject matter provides an increase in the throughput and translatability of magnetic nanoparticle-based artificial antigen presenting cells (aAPCs) to enrich and expand (E+E) murine or human antigen-specific T cells. In some aspects, the presently disclosed subject matter streamlines enrichment, expansion, and aAPC production processes by enriching CD8+ T cells directly from unpurified immune cells, increasing parallel processing capacity of aAPCs in a 96-well plate format, and designing an adaptive aAPC that enables multiplexed aAPC construction for E+E and detection. In representative embodiments, the presently disclosed adaptive platforms were applied to process and detect CD8+ T cells specific for rare cancer neoantigens, commensal bacterial cross-reactive epitopes, and human viral and melanoma antigens. These innovations dramatically increase the multiplexing ability and decrease the barrier to adopting the platform for investigating antigen-specific T cell responses.

More particularly, the presently disclosed methods enrich and expand (E+E) rare antigen-specific T cells with MHC and costimulatory molecules (such as anti-CD28). Perica et al., ACS Nano (2015); Hickey et al, Biomaterials (2018); Kosmides et al., Nano Lett. 2018. In contrast to methods known in the art, the presently disclosed platform eliminates the requirement of costly cell isolation kits. Eliminating this isolation step unexpectedly also simultaneously enhances CD8+ T cell activation apparently from the presence of additional immune cells, e.g., CD4+ T cells.

This technology is further adapted to be higher throughput with the capability of processing multiple antigen-specific T cells in parallel. Fluorescent magnetic nanoparticles also can be used to create a new adaptive detection bead that enables parallelized detection reagent production. The utility of the presently disclosed system is demonstrated through multiplexed expansion of murine antigen-specific T cells, including commensal bacterial cross-reactive CD8+ T cells (SVY), detection of rare, low affinity neoantigen CD8+ T cells, and expansion of human viral and tumor-specific CD8+ T cells. The simplicity of these technologies makes them easy to adopt by non-specialists and provides a high-throughput workflow for identification and analysis of antigen-specific T cell responses.

### 1.2 Results and Discussion

1.2.1 Antigen-Specific T Cell Enrichment and Expansion from Splenocytes

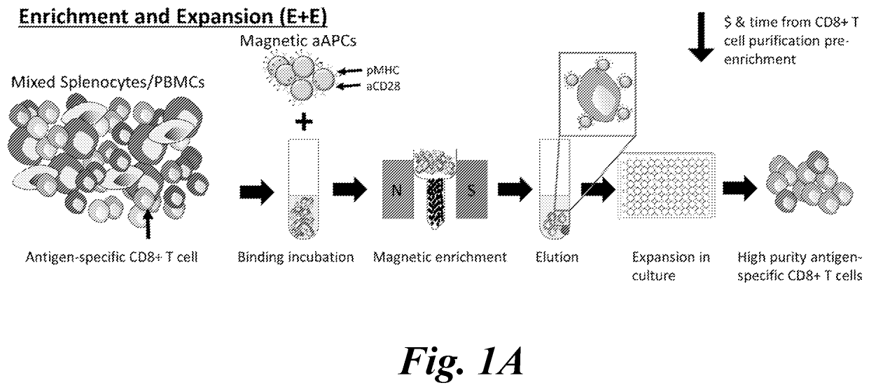

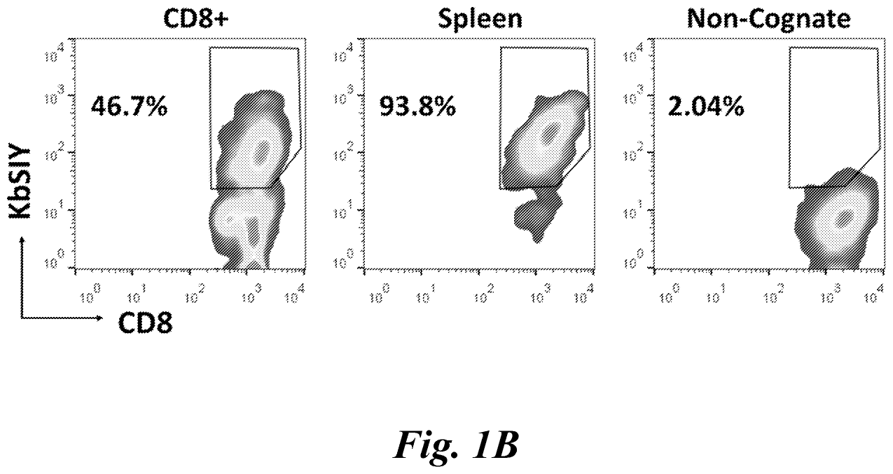

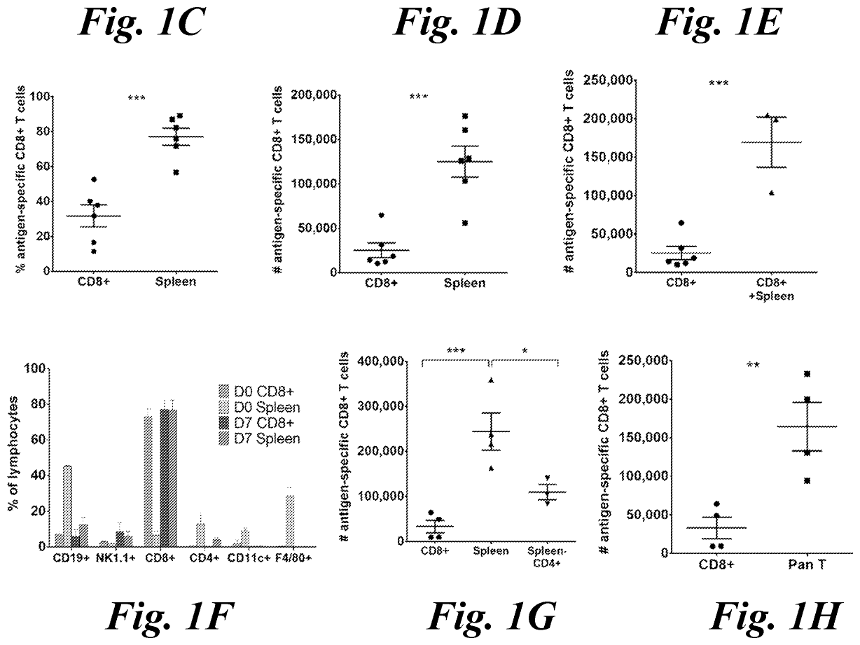

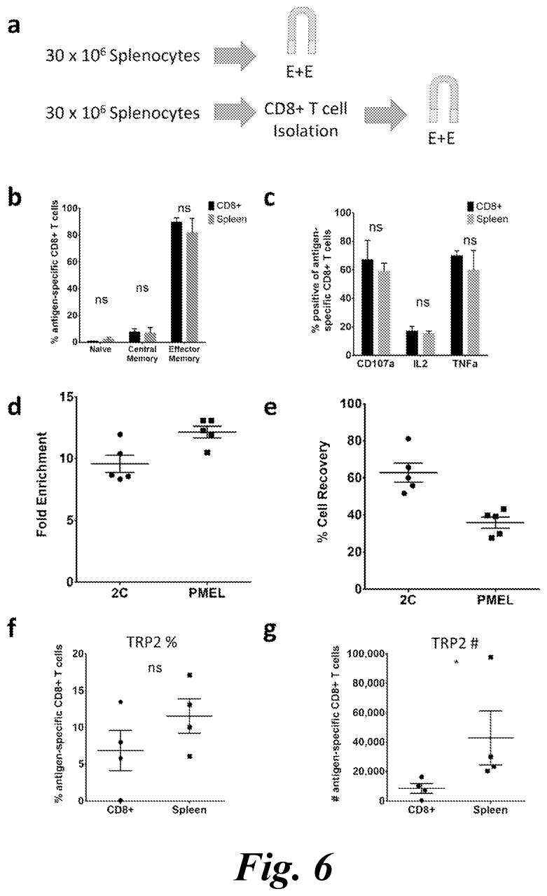

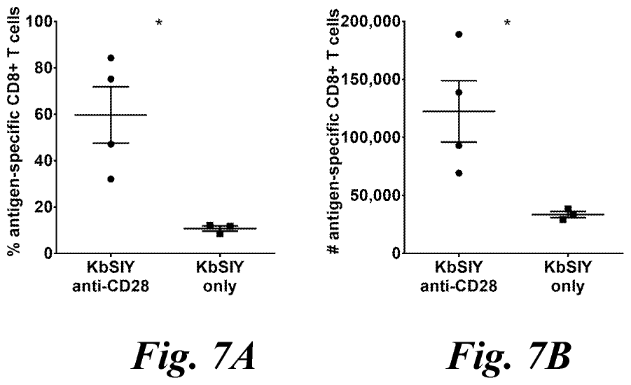

Enrichment and Expansion (E+E) protocols known in the art require CD8+ isolation prior to adding magnetic nanoparticle aAPCs for enriching T cells. Perica et al., ACS Nano (2015). To streamline this process, antigen-specific CD8+ T cells were enriched and then expanded directly from unpurified splenocytes (FIG. 1A, FIG. 6A). Interestingly, the E+E from splenocytes dramatically improved the purity of the expanded population post-expansion (FIG. 1B). More particularly, the percent and number of SIY-specific CD8+ T cells increased by two- (FIG. 1C) and five-fold (FIG. 1D), respectively, after seven days of expansion while phenotype and function were conserved (FIG. 6B, FIG. 6C). This observation was not due to differences in efficiency of enrichment (FIG. 6D) or percent recovery (FIG. 6E) between purified CD8+ T cell and splenocyte populations; nor was it unique to SIY, as similar results were observed when expanding for endogenous antigen TRP2 (FIG. 6F, FIG. 6G). As with E+E from CD8+ T cells, Kosmides et al., Nano Lett. (2018), optimal E+E from splenocytes required anti-CD28 to be present on aAPCs (FIG. 7A-FIG. 7B).

Without wishing to be bound to any one particular theory, it was thought that this boost in output was driven by the presence of non-CD8+ T cells. To investigate this hypothesis, SIY-specific CD8+ T cells were enriched from a purified CD8+ population with the presently disclosed aAPCs and splenocytes were then added post-enrichment, functionally diluting CD8+ T cells back to their initial frequency with non-CD8+ T cells. This approach increased the number of antigen-specific cells by six-fold on day 7 (FIG. 1E), confirming the initial hypothesis.

Which non-CD8+ T cell populations contributed to enhanced CD8+ T cell expansion was then investigated because splenocyte E+E starting populations (post-aAPC enrichment) also included B cells (CD19+), NK cells (NK1.1+), CD4+ T cells, dendritic cells (CD11c+), and macrophages (F4/80+), despite converging to a relatively homogenous CD8+ population by day 7 (FIG. 1F). It was thought that the presence of CD4+ T cells could drive this boost, considering their natural roles in the priming of naïve CD8+ T cells. Inaba et al., J. Exp. Med. (1987); Novy et al., J. Immunol. (2007).

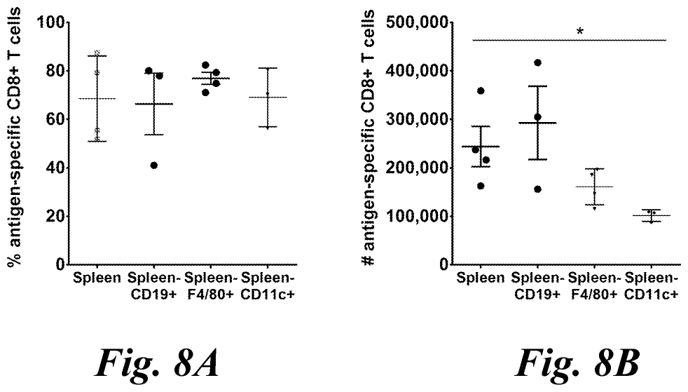

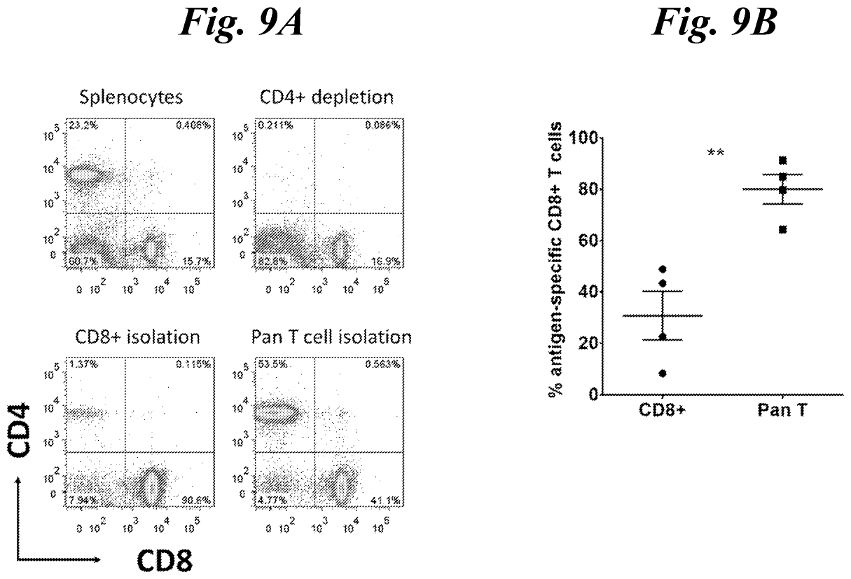

To address this hypothesis, CD4+ T cells were depleted (FIG. 9A) pre-enrichment from splenocytes. It was found that depletion of CD4+ T cells significantly decreased the number of antigen-specific cells on day 7 (FIG. 1G). Finally, whether CD4+ T cells alone were sufficient to improve the output was investigated by performing E+E on a cell population purified with a Pan T cell isolation kit. Interestingly, it was found that using a Pan T cell isolate (FIG. 9A) significantly boosted the frequency (FIG. 9B) and number (FIG. 1H) of antigen-specific T cells. Among endogenous antigen presenting cells, such as B cells, macrophages, and dendritic cells, only depletion of dendritic cells pre-enrichment significantly reduced the output from splenocyte E+Es (FIG. 8A—FIG. 8B).

Thus, this approach of isolating from splenocytes represents not only a cost, time, and technical advantage, but also demonstrates the importance of additional CD4+ T cell support during CD8+ activation. Since MHC class I aAPCs only stimulate CD8+ T cells ex vivo, the presence of CD4+ T cells was not expected to provide significant benefit. Perica et al., ACS Nano (2015). The presently disclosed results suggest the opposite and future work is warranted to fully understand this interaction. Finally, this finding also supports the need for development of MHC class II aAPCs, where simultaneous expansion of antigen-specific CD4+ and CD8+ T cells may be mutually beneficial to ex vivo expansion.

### 1.2.2 High Throughput Enrichment and Expansion

Previous efforts for enrichment and expansion of antigen-specific T cells have included the use of 50-nm aAPCs, Perica et al., ACS Nano (2015), which require specially-produced magnetic columns to produce magnetic fields strong enough to retain labeled cells. This requirement limits the throughput and adaptability of the protocol. How the size of the aAPC impacts both T cell activation, Hickey et al., Nano Lett. (2017), and enrichment, Hickey et al., Biomaterials (2018) was studied previously, and it was found that particles close to 300 nm were most efficient at activating and enriching antigen-specific T cells. These 300-nm aAPCs can be magnetically isolated with weaker magnetic fields, such as conventional permanent magnet including, but not limited to, neodymium magnets, and thus can be adapted to a 96-well plate format (FIG. 2A).

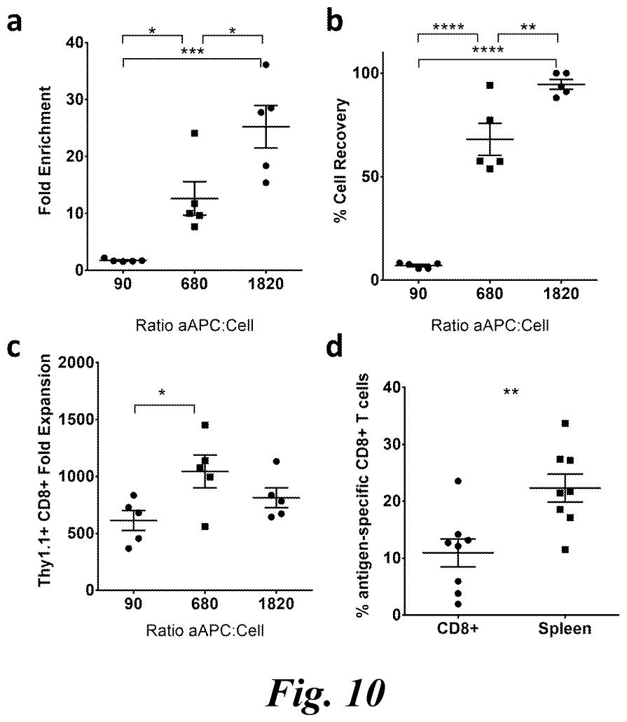

It also was found that nanoparticle concentration is a key factor influencing the optimal E+E of antigen-specific T cells. Hickey et al., Biomaterials (2018). Accordingly, the concentrations of aAPC E+E were optimized with this new particle size (300 nm), starting cell populations (splenocytes), and magnet format (96-well plate neodymium magnet) for enrichment (FIG. 10A), cell recovery (FIG. 10B), and cell expansion (FIG. 10C). This 96-well plate set-up was then used to perform E+Es on endogenous antigen-specific T cells from a wild-type B6 mouse, and confirming previous results where an increase in expansion of antigen-specific T cells was observed previously by starting from a population of splenocytes compared to purified CD8+ T cells (FIG. 2B, FIG. 10D). All of the following E+E experiments were conducted with this 96-well plate from a starting population of splenocytes.

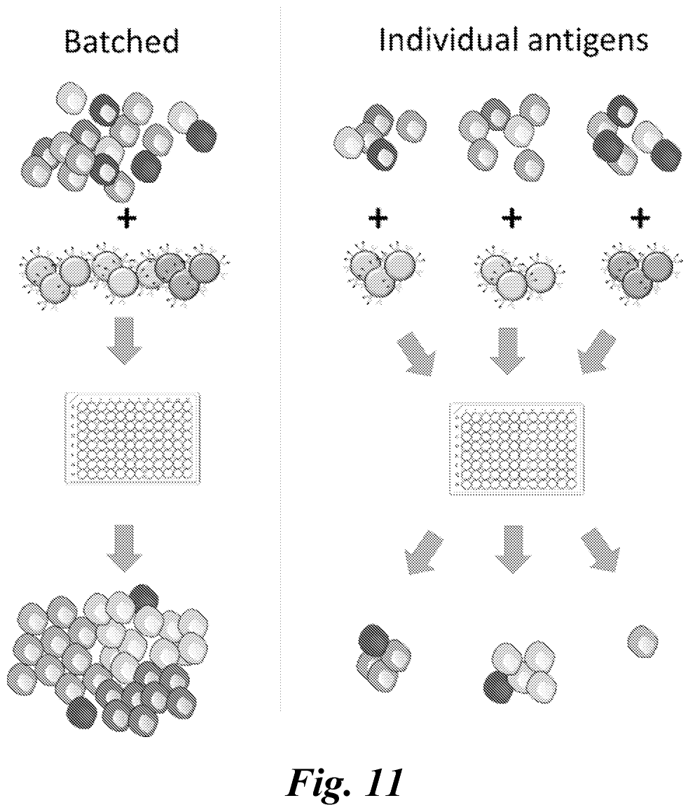

To test its ability for parallel processing, four distinct antigen-loaded aAPCs (gp100, SIY, TRP2, OVA) were generated and E+Es were performed on endogenous T cells with the 96-well plate format. Batched versus separate enrichments were compared (FIG. 11) and both conditions were found to produce similar percentages (FIG. 2C) with slightly higher numbers of antigen-specific T cells in batched conditions, though not statistically significant (FIG. 2D). This observation suggests that either configuration could be used to identify antigen-specific T cells depending on the availability of samples or number of combinatorial staining reagents.

1.2.3 Adaptive aAPCs to Enrich and Expand T Cells with Multiple Antigen Specificities

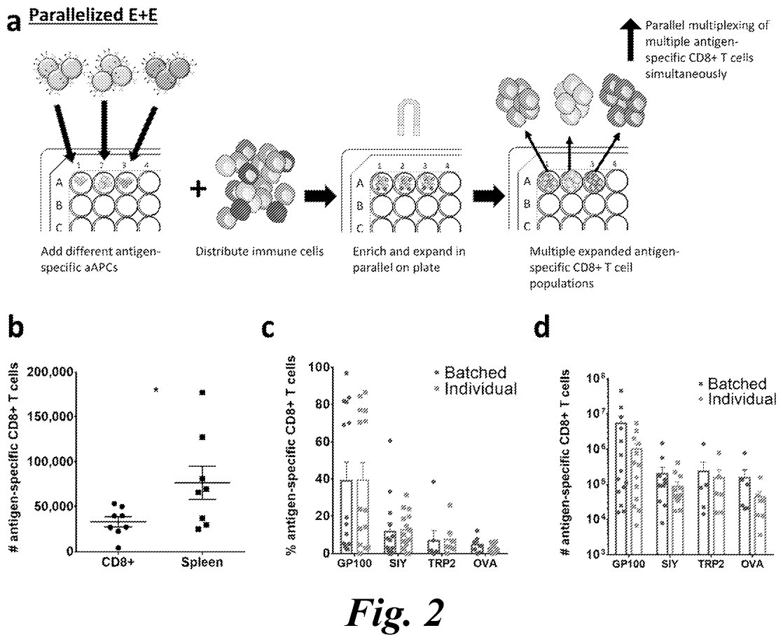

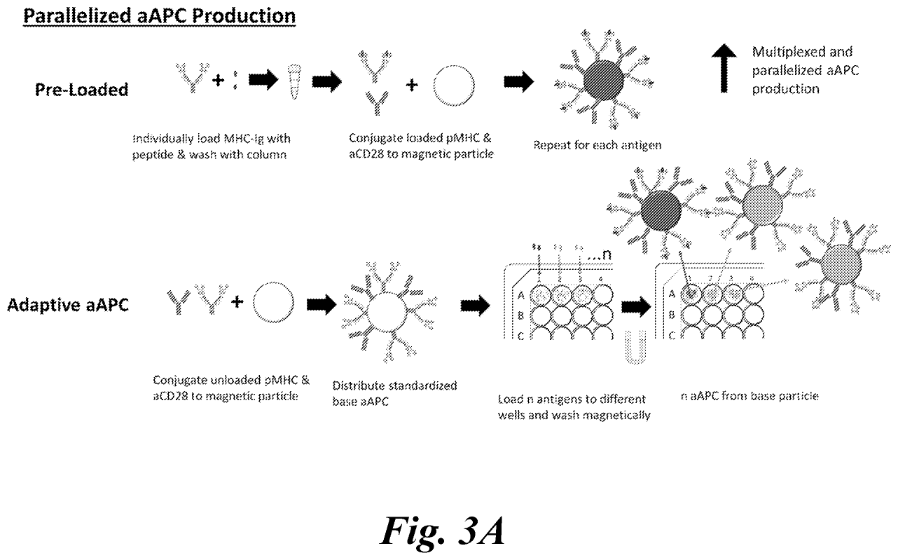

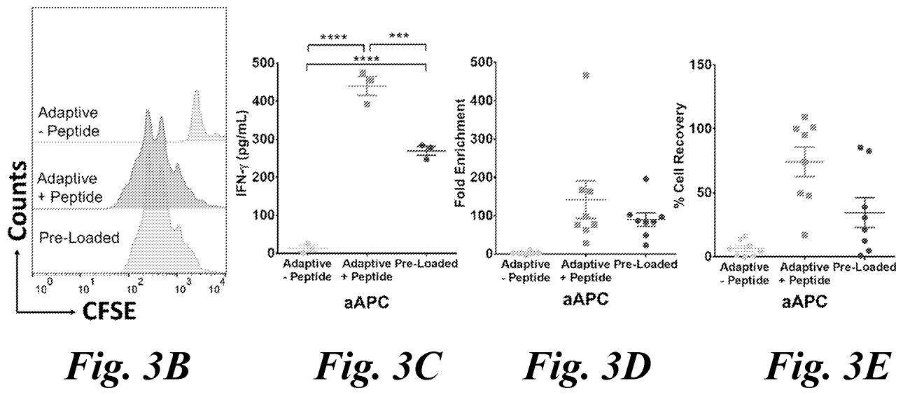

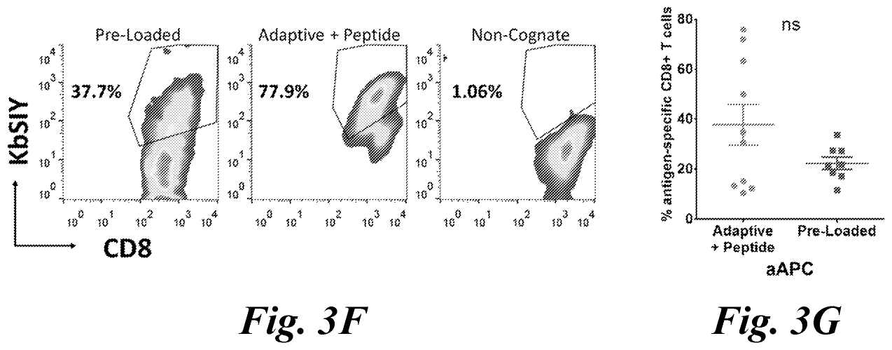

While the 96-well plate format provides a convenient, high-throughput approach to enrich antigen-specific T cells, creating individual, antigen-specific aAPCs can be labor- and reagent-intensive. Rather than loading and then coupling dimeric MHC-Ig (pre-loaded), unloaded dimeric MHCs were conjugated directly to magnetic nanoparticles creating an adaptive aAPC that requires a one-step particle synthesis and standardizes aAPC reagents (FIG. 3A). These peptide-less aAPCs can be aliquoted into individual wells on 96-well plates and efficiently loaded through incubation with peptides for multiplexing the number of antigens that can be probed at a time.

To confirm the function of adaptive aAPCs, their efficiency in stimulating antigen-specific T cells was evaluated. T cells proliferated at comparable rates with both adaptive (+peptide) and pre-loaded aAPCs by CFSE dilution assay after 3 days of culture (FIG. 3B). In contrast, unloaded adaptive aAPCs (-peptide) did not result in any measurable activation, demonstrating both the specificity and efficiency of the adaptive peptide loading process. Additionally, the functional response of 2C cells to adaptively loaded aAPCs as measured by IFN-γ secretion was significantly higher as compared to pre-loaded KbSIY aAPCs or unloaded adaptive aAPCs (FIG. 3C).

In addition, the ability of these peptide-loaded adaptive aAPCs to bind and enrich antigen-specific T cells was tested. In a doped enrichment experiment, similar to higher levels of fold enrichment (FIG. 3D) and recovery (FIG. 3E) of target cells were observed with the peptide-loaded adaptive aAPCs compared to pre-loaded aAPCs, while unloaded adaptive aAPCs produced no measurable antigen-specific enrichment. Additionally, after E+E of endogenous antigen-specific T cells, peptide-loaded adaptive aAPCs generated comparable levels of expansion of antigen-specific T cells by day 7 to pre-loaded aAPCs (FIG. 3F, FIG. 3g). Together, these results demonstrate that adaptive aAPCs provide a facile method for parallel production of multiple antigen-specific aAPCs in a single process, all while using smaller amounts of costly pMHC.

### 1.2.4 Adaptive Detection Beads for High-Throughput Antigen-Specific T Cell Detection

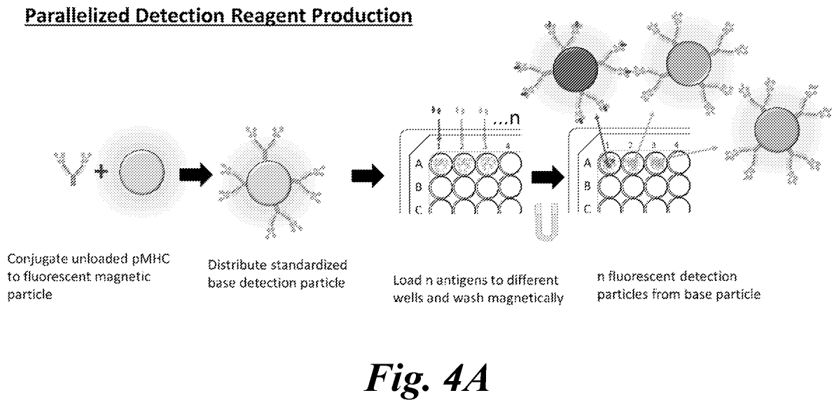

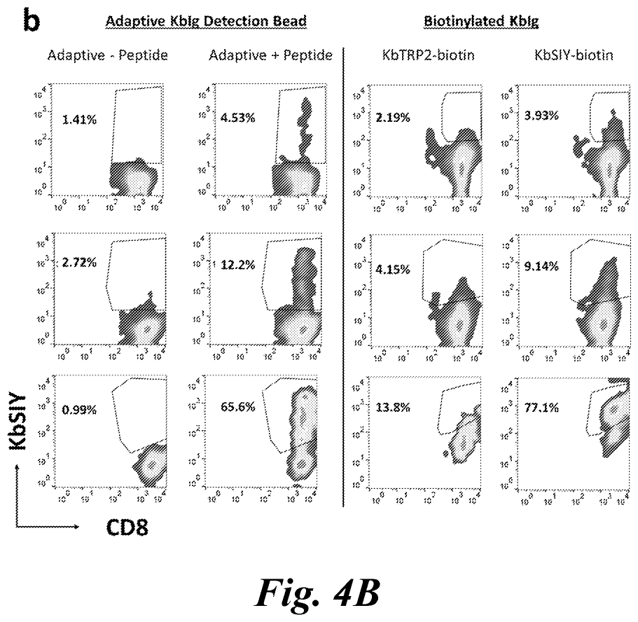

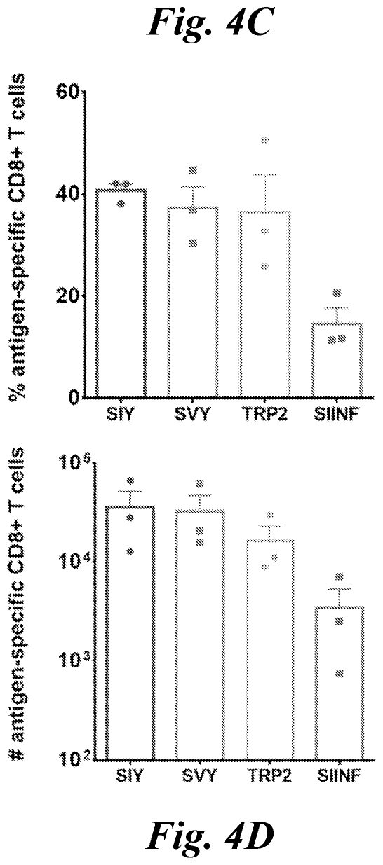

A multiplexed process for enriching and expanding T cells with many antigen specificities, necessitates new technologies to increase multiplexing of detecting antigen-specificity post-expansion. Current methods to produce detection of pMHC complexes require biotinylation of dimeric or tetrameric MHC before loading and washing with each individual antigen. This approach is a labor-intensive process, resulting in losses of expensive MHC protein. An adaptive detection bead was produced using fluorescently labelled magnetic particles conjugated with unloaded MHC-Ig (FIG. 4A). This approach builds off of the results shown in FIG. 3, confirming that peptide-loaded adaptive aAPCs work well for antigen-specific binding, and previous work showing multivalent pMHC on the aAPC surface has high cognate affinity interactions with CD8+ T cells. Hickey et al., Biomaterials (2018).

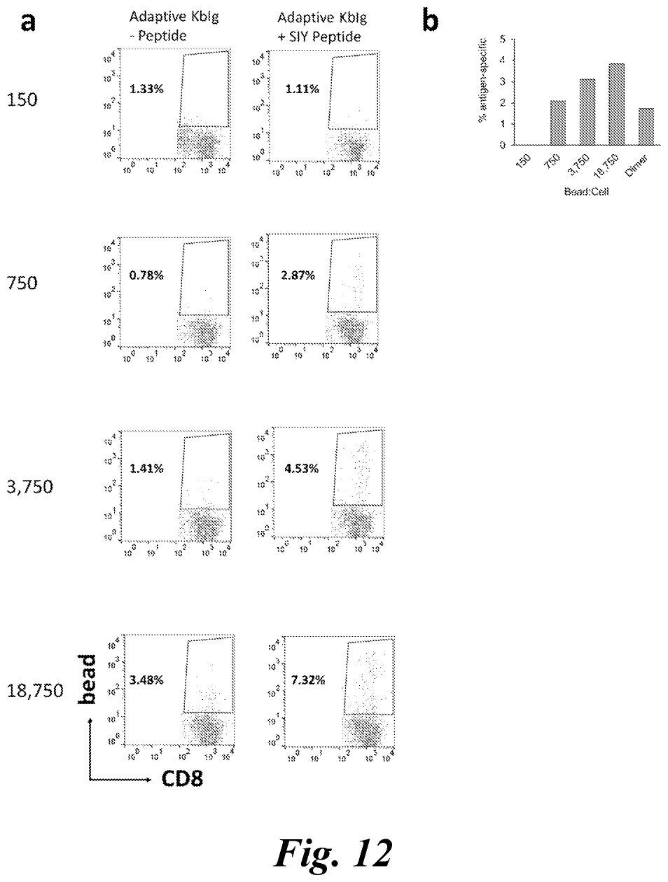

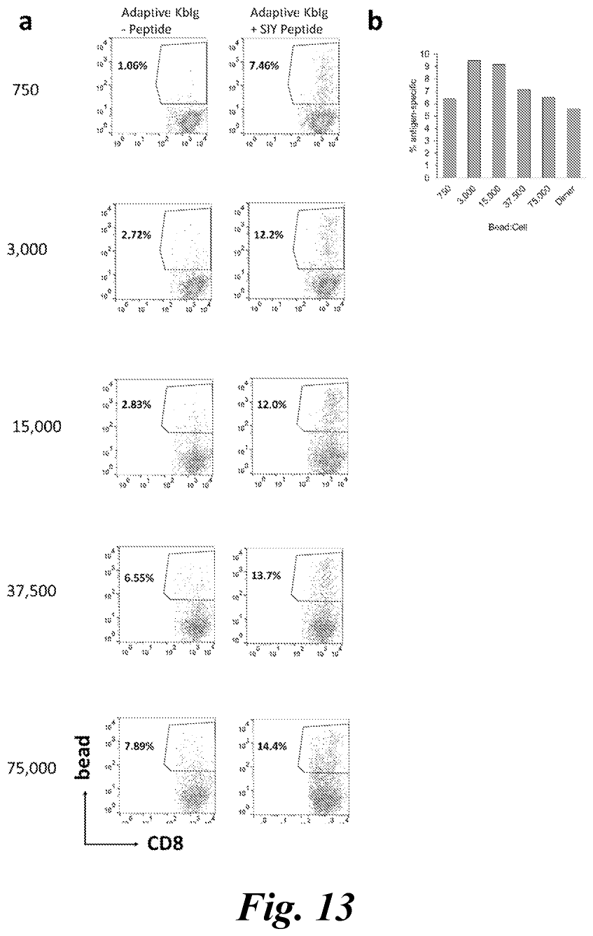

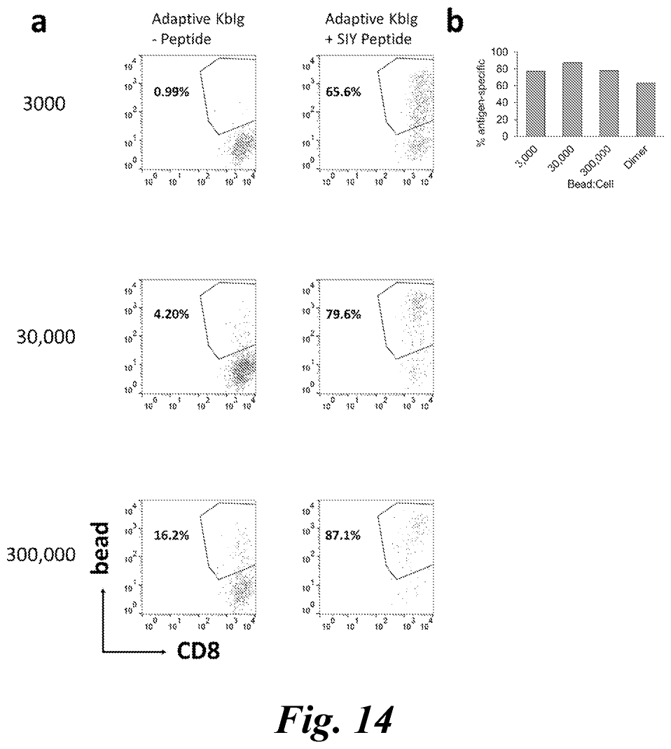

To test the efficacy of these adaptive detection beads, the beads were incubated with SIY peptide and then used to identify endogenous antigen-specific T cells at low, intermediate, and high antigen-specific frequencies post E+E. Compared to a traditional biotinylated dimeric pMHC, the adaptive detection beads efficiently detected antigen-specific T cells with relatively low background (FIG. 4B). Optimal fluorescent bead dose was found at a bead to cell ratio of 3,000 for each of the antigen-specific frequencies (FIG. 12-FIG. 14). Thus, these fluorescent magnetic detection beads can be customized with target peptides and easily added for sensitive staining of antigen-specific T cells. Compared to traditional staining reagents, like dimers or tetramers, this capability establishes a universal base particle that enables adaptation to antigens of interest, as well as 96-well plate-based parallel processing.

One area of increasing interest is CD8+ T cell cross-reactivity. It has previously been investigated how a gut microbiota-derived antigen SVY leads to expansion of CD8+ T cells cross-reactive for the SIY antigen, with demonstrated increased SIY+ tumor killing (Bessell et. al, in press). Investigation of CD8+ T cell cross-reactivity would benefit from a platform that enables simultaneous identification of multiple antigen-specific T cells, including antigenic controls.

Further, having only one variable changing as the antigen that is loaded would provide a more reliable comparison and reproducible evaluation of properties of cross-reactivity, where there may be variability between batches of individually produced staining reagents.

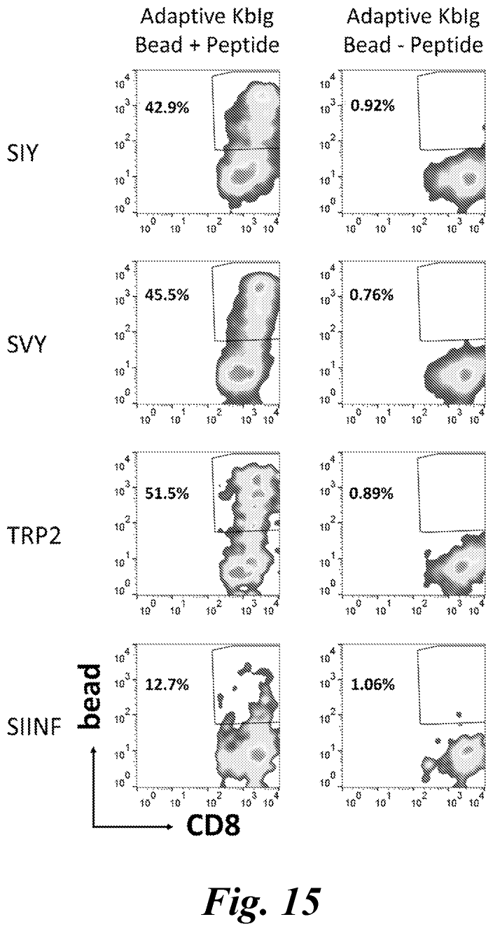

To demonstrate this capability, adaptive aAPCs were loaded on Day 0 with SIY (antigen of B16-SIY melanoma tumor), SVY (cross-reactive Bifido bacterium antigen), TRP2 (B16 endogenous melanoma antigen), and SIINF (ovalbumin model antigen). Following E+E, on Day 7, adaptive detection beads were loaded with the same four peptide and successfully detected T cells specific to all four antigens simultaneously, with antigen-specific percentages between 10 and 40% (FIG. 4C-FIG. 4D, FIG. 15). In summary, the presently disclosed subject matter provides an adaptive nanoparticle platform for detection of CD8+ T cells with a range of antigen-specificities, which complements the presently disclosed adaptive aAPCs that allow for multiplexed E+E.

1.2.5 Adaptive aAPC Platform for Cancer Neoantigens and Human Antigen-Specific T Cells

The presently disclosed high-throughput and adaptive detection platform for identifying antigen-specific T cells extends the ability to investigate multiple candidate antigens, which will be beneficial for cancer neoantigens. Neoantigens are processed and presented peptides derived from mutated tumor proteins to which the immune system has not been tolerized. Thus, they represent unique and specific immune cell targets for the tumor. Yarchoan et al., Nat. Rev. Cancer (2017); Schumacher and Schreiber, Science (2015). Treatments targeting neoantigens have led to dramatic clinical results in both adoptive immunotherapy and tumor vaccines. Abiko et al., Br. J. Cancer (2015); Sahin et al., Nature (2017).

Neoantigen-specific therapies, however, have been limited because of challenges in identifying antigen-specific T cell responses. With hundreds to thousands of potential antigen candidates for each patient, current techniques can only examine a few antigen-specific responses at one time and thus rely heavily on imperfect prediction algorithms, Gonzalez et al., Semin. Cancer Biol. (2018); Sarkizova and Hacohen, Nature (2017), Topalian et al., Nat. Rev. Cancer (2016), and are labor intensive. Adaptive nanoparticle platforms overcome many of these challenges and are therefore poised to identify neoantigen-specific cells for understanding these responses and for designing better, more targeted, immunotherapies.

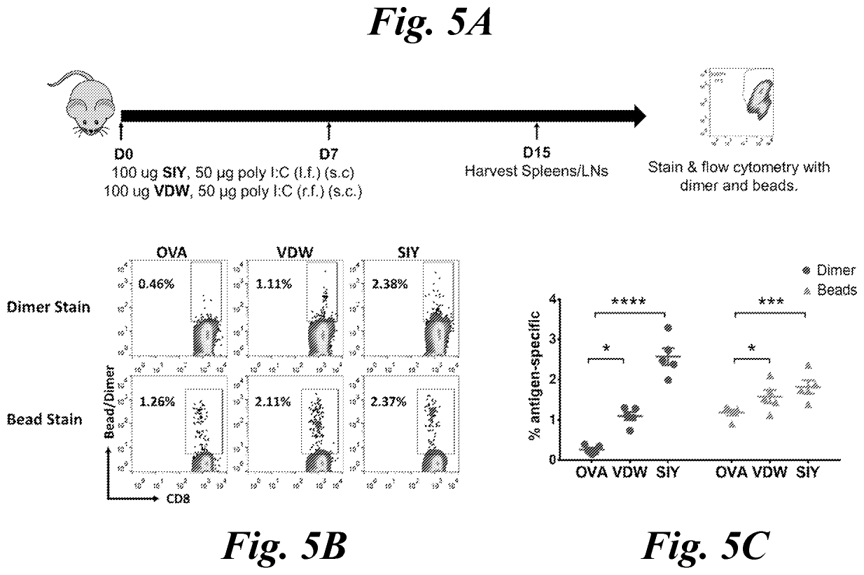

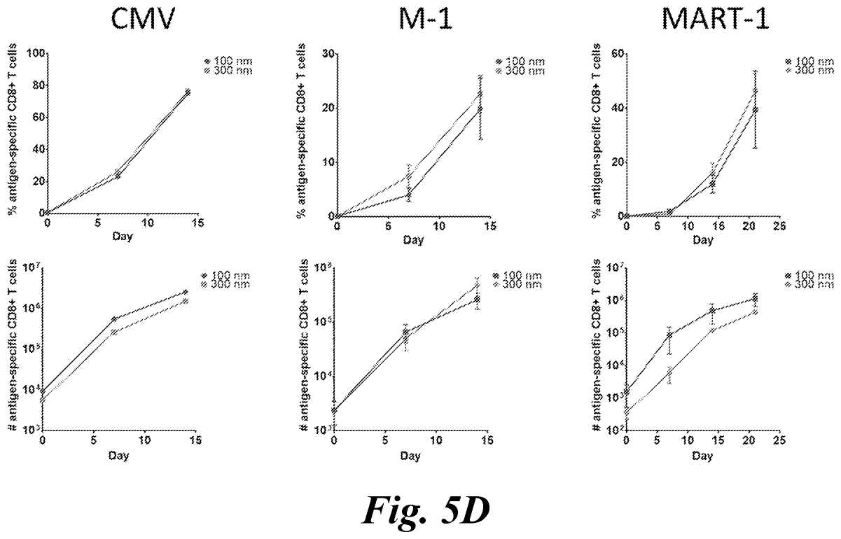

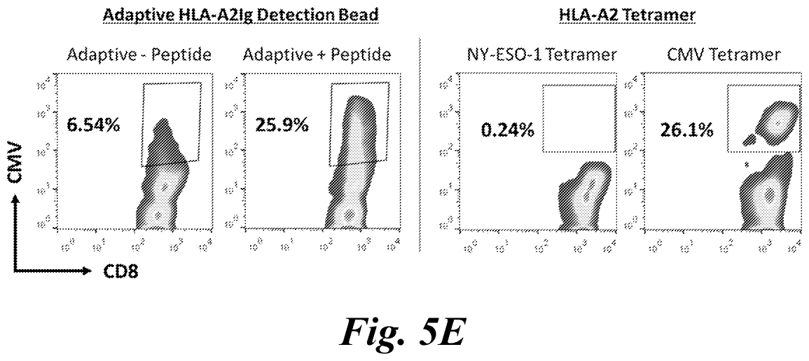

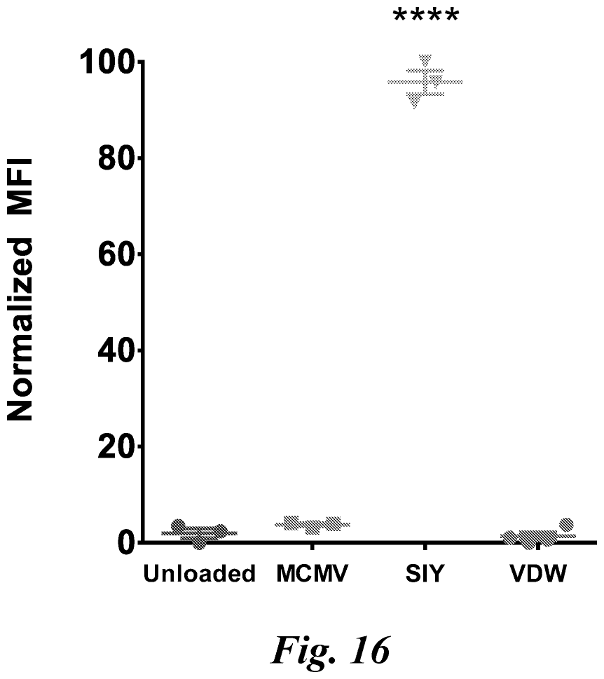

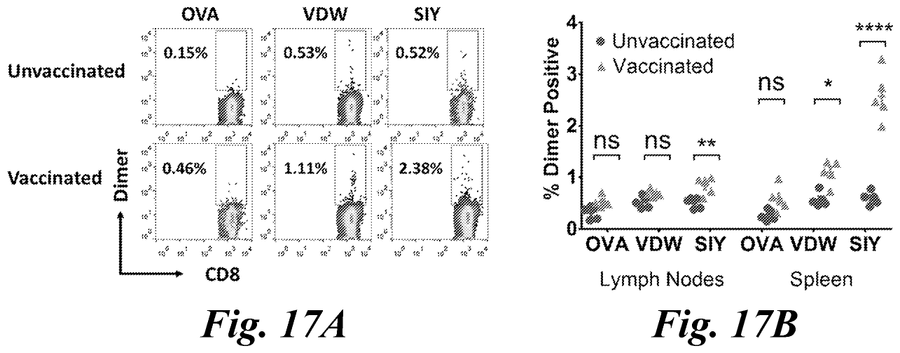

To study whether the presently disclosed platform could be used to detect neoantigen-specific CD8+ T cells, the efficacy of staining a B16-F10-derived neoepitope, Kif18b₇₃₅₋₇₄₅, VDWENVSPEL (VDW), which was previously shown to be immunogenic was analyzed. Castle et al., Cancer Res. (2012). In comparison to the SIY peptide, VDW has low affinity for Kb (FIG. 16), allowing one to assess whether the presently disclosed detection platform can be used for staining low affinity peptides. To expand VDW-specific T cells, mice were vaccinated with poly I:C and SIY and VDW peptide (FIG. 5A). Castle et al., Cancer Res. (2012). Comparison of dimer staining for KbSIY and KbVDW pre- and post-vaccination showed significant increases in both SIY and VDW-specific CD8+ T cells (FIG. 17). Particle stains for SIY and VDW from splenocytes of vaccinated mice were then compared and a significantly greater particle staining of SIY and VDW specific T cells over background was observed (FIG. 5B, FIG. 5C). These results confirmed that the presently disclosed adaptive detection beads were capable of staining neoantigen-specific CD8+ T cells.

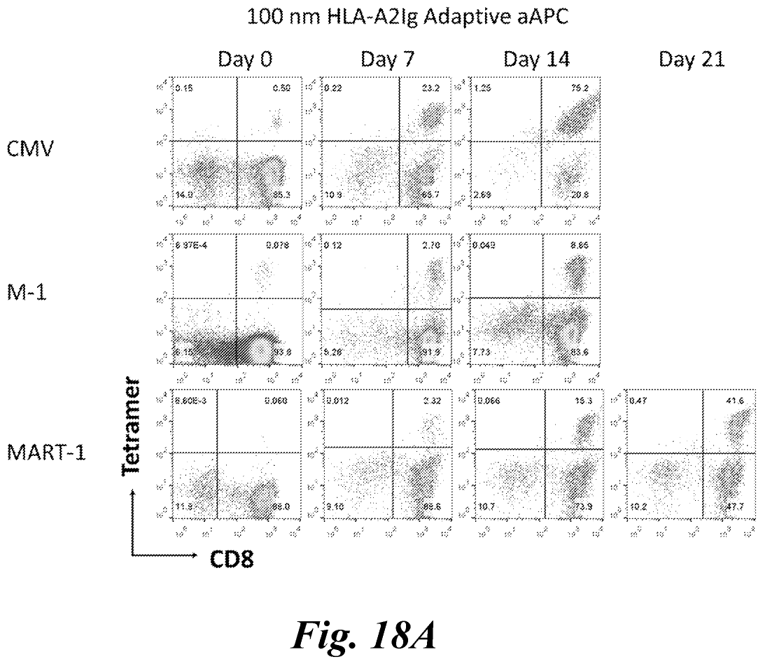

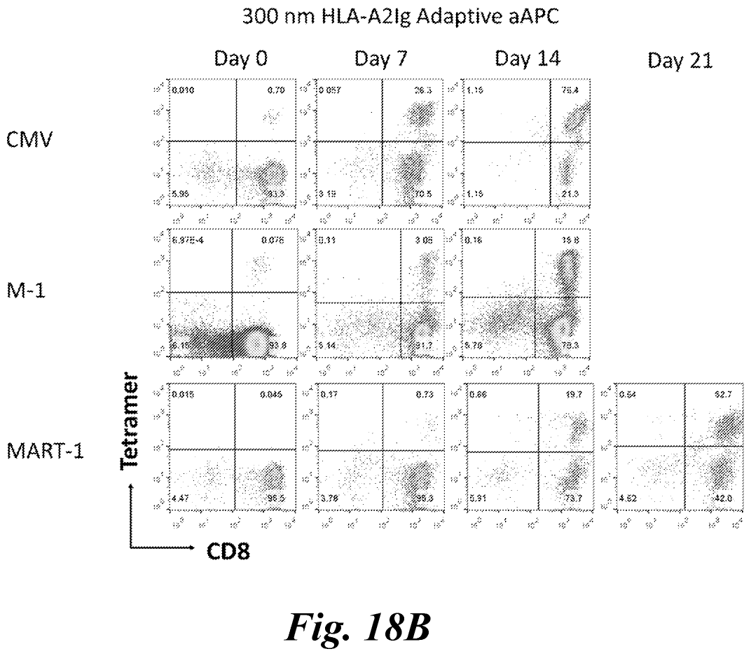

To understand the clinical utility of these platforms, the presently disclosed multiplexed adaptive aAPCs and detection beads were applied to human antigen-specific CD8+ T cells. Effective expansion was observed with both 100- and 300-nm adaptive aAPCs pulsed with viral antigens (CMV and M-1), as well as melanoma antigens (MART-1) (FIG. 5D, FIG. 18). Specifically, precursor frequencies increasing from 0.02-0.5% to 20-70% were observed by Days 14 and 21 (FIG. 5D, top panel, FIG. 18). Similarly, log-fold number increases in total antigen-specific CD8+ T cells were obtained by Days 14 and 21, ending up with 10⁵-10⁶ antigen-specific CD8+ T cells from precursor numbers between 100 and 1,000, representing nearly a 200 fold increase by Day 14 for CMV and M-1 and a 500-2000-fold-increase by Day 21 for MART-1 (FIG. 5D, bottom panel). Finally, to determine if the presently disclosed detection platform could be applied to endogenous human-specific CD8+ T cells, a similar approach to synthesize fluorescent HLA-A2Ig detection beads and then load with peptide of interest was followed. It was found that the detection beads had similar efficacy at staining endogenous CMV-specific CD8+ T cells as CMV-specific tetramers (FIG. 5E). This observation indicates the presently disclosed system can be applied to studying and discovering human antigen-specific CD8+ T cells.

### 1.3. Summary

The presently disclosed subject matter provides magnetic nanoparticle platforms to substantially improve expansion and detection of antigen-specific T cells and extended the process throughput and adaptability. First, the need to enrich and expand antigen-specific T cells from purified CD8+ T cell populations was eliminated. This characteristic decreased the total cost and time needed to perform the assay and resulted in significant increases in both the numbers and percentages of antigen-specific CD8+ T cells. Second, the size of the aAPCs was modified to 300 nm to enable parallel processing of multiple antigen-specific aAPC enrichments on a 96-well plate. Third, to further increase multiplexing and to create a standardized base particle, an adaptive aAPC, where the MHC-Ig was conjugated to the surface of the particle and subsequently divided and loaded with a range of peptides, was created. Fourth, a fluorescent, magnetic adaptive detection bead that can be loaded with a range of peptides to parallelize antigen-specific staining reagent production was created. Each of these engineered technologies and streamlined processes for E+E of antigen-specific T cells overcomes critical difficulties in processing and identifying antigen-specific T cells. More particularly, the presently disclosed technique is (a) sensitive, as it amplifies signal from both expanding and staining rare antigen-specific CD8+ T cells; (b) high-throughput, as the combination of the 96-well plate E+E format, adaptive aAPCs, and adaptive detection beads allow multiplexing for isolation and analysis of antigen-specific T cells; and (c) easy to use, as CD8+ T cell isolation kits are removed and nanoparticle reagents are easily customizable.

The presently disclosed technology was applied to isolate and identify a range of antigen-specific CD8+ T cells across disease, species, TCR affinity, and number of unique antigens. CD8+ T cells specific for low-affinity cancer neoantigens were examined, which demonstrates its versatility and ability to discover unknown antigen-specific CD8+ T cells to new antigens. CD8+ T cells specific toward several antigens simultaneously were evaluated, which facilitates the ability to examine cross-reactivity in the same sample. Finally, the presently disclosed tool was evaluated with respect to human infectious disease and melanoma antigens, which demonstrates direct clinical relevance either as a diagnostic or therapeutic. It is anticipated that the throughput and parallel processing capabilities of the presently disclosed adaptive nanoparticle system will lend it to be adopted into other high-throughput assays, such as single-cell RNA sequencing, which would enable unique TCR-antigen combination analysis. In summary, the presently disclosed subject matter facilitates adoption of both the platform and process to study unprecedented numbers and types of antigen-specific T cell responses in infectious disease, autoimmunity, allergy, and cancer.

### 1.4. Experimental

### 1.4.1 Mice

B6, 2C, and PMEL transgenic mice were maintained per guidelines approved by the Johns Hopkins University's Institutional Review Board. C57BL/6J mice were purchased from Jackson Laboratories (Bar Harbor, Me., USA). 2C T cell receptor transgenic mice were kept as heterozygotes by breeding on a C57BL/6J background.

### 1.4.2 MHC-Ig and Peptides

Soluble MHC-Ig dimers loaded with peptides (“Pre-loading”) including DbIg, KbIg, and A2Ig were produced in-house as described. Kosmides et al., Nano Lett. (2018); Hickey et al., JoVE (Journal Vis. Exp.) (2018). Peptides for experiments used include: GP100: KVPRNQDWL (SEQ ID NO: 1), SIY: SIYRYYGL(SEQ ID NO: 2), SVY: SIYRYYGL(SEQ ID NO: 3), OVA: SIINFEKL(SEQ ID NO: 4), TRP2: SVYDFFVWL(SEQ ID NO: 5), VDW: VDWENVSPEL(SEQ ID NO: 6), MCMV: YPHFMPTNL(SEQ ID NO: 7), CMV: NLVPMVATV(SEQ ID NO: 8), M1: GILGFVFTL(SEQ ID NO: 9), MART-1: ELAGIGILTV(SEQ ID NO: 10). Peptides were purchased from GenScript (New Jersey, USA). 2C T cell transgenic mice are cognate for SIY peptide loaded into KbIg and PMEL transgenic mice are cognate for GP100 peptide loaded into DbIg.

### 1.4.3 Pre-Loaded Artificial Antigen Presenting Cells Production

Artificial antigen presenting cells (aAPC) were produced in-house as described. Hickey et al., JoVE (Journal Vis. Exp.) (2018); Hickey et al., Biomaterials (2018). Briefly, loaded antigen-specific dimeric MHC-Ig and equimolar anti-CD28, clone 37.51 (BioXCell. West Bebanon, N.H., USA) were conjugated to the surface of magnetic particles functionalized with NHS surface groups at a based particle size of 200 nm (OceanNanotech, Springdale, Ark., USA) per the manufacturer's recommendations.

### 1.4.4 Adaptive Artificial Antigen Presenting Cells Production

For Adaptive aAPCs, MHC-Ig was conjugated to particles (with the same method as Pre-loaded) except without previously loading in a specific peptide.

### 1.4.5 Detection Bead Production

For detection beads, BNF-Starch-greenF 100-nm magnetic particles with amine surface groups (Micromod, Rostock, Germany) were functionalized with Sulfo-SMCC (Proteochem, Hurricane, Utah, USA) and dimeric MHC-Ig was thiolated with Traut's reagent (2-iminothiolane) (Sigma Aldrich, St. Louis, Mo., USA) and then mixed with the functionalized particles per the manufacturer's recommendations. For adaptive detection beads, MHC-Ig was conjugated to particles without previously loading in a specific peptide.

### 1.4.6 Artificial Antigen Presenting Cell Characterization

The amount of protein conjugated successfully to the surface of the particles was quantified through fluorescent staining. The amount of MHC-Ig was quantified by staining with FITC-conjugated rat anti-mouse Ig λ1, λ2, λ3 light chain, clone R26-46 (BD Biosciences, San Jose, Calif., USA), and the amount of anti-CD28 was quantified by staining with FITC-conjugated mouse anti-Armenian Syrian hamster IgG, clone G192-1 (BD Biosciences). Particles were stained with 1 μL of the antibody for 1 h at 4° C., washed three times, and then fluorescence was read on Synergy HTX Multi-mode florescent plate reader (BioTek, Winooski, Vt., USA). Protein was quantified by comparison to fluorescent standard curve of staining antibodies, and particle number was quantified by absorbance using a spectrophotometer at a wavelength of 405 nm.

1.4.7 Adaptive aAPCs and Detection Bead Peptide Loading

To load Adaptive aAPCs or Detection Beads, an aliquot of aAPCs (1.5×10¹⁰ particles) or Detection Beads (3.8×10¹⁰ particles) was aliquoted into a final volume of 1004 of PBS in a 96-U bottomed plate. Then 1 μg of peptide was added to the particles overnight at 4° C. The particles were washed three times on the “Ring” magnet with 200 μL of PBS and immediately used. 1.4.8 Supplemented Media and T Cell Growth Factor

Supplemented media (B′ Media) was made with PBS buffer and 0.5% bovine serum albumin (BSA) (Gemini, Sacramento, Calif.) and 2 mM EDTA. The T cell growth factor (TCGF) was made with RPMI 1640 media with glutamine, lx non-essential amino acids, 1 mM sodium pyruvate, 0.4×vitamin solution, 92 μM 2-mercaptoethanol, 10 μM ciprofloxacin and 10% fetal bovine serum (FBS) (Atlanta Biologicals, Flowery Branch, Ga.).

### 1.4.9 Specific Cell Isolation and Depletion

Murine cells were obtained from adult female and male mouse lymph nodes and spleens. Obtained cells were treated with ACK lysing buffer to lyse red blood cells and filtered through cell strainers to isolate splenocytes. PBMCs from healthy human donors were isolated by Ficoll-Paque PLUS gradient centrifugation (GE Healthcare, Chicago, Ill., USA). For isolating CD8+ T lymphocytes, CD4+ T lymphocytes, Pan T cells, these cells were isolated from splenocytes or PBMCs by negative selection using CD8+, CD4+, and Pan T cell isolation kits and magnetic columns from Miltenyi Biotech (Auburn, Calif., USA) according to the manufacturer's protocol. Memory CD8+ T cells were not depleted for all CD8+ isolations, in order to maintain consistency with CD8+ populations we would encounter in isolating antigen-specific T cells from splenocyte or PBMC sources. To deplete specific cell populations, biotinylated antibody was added to splenocytes for 5 min at 4° C., followed by ratio of anti-biotin magnetic beads consistent with previous manufacturer recommended amounts from isolation kits (Miltenyi Biotech); for CD4+ T cells, clone Gk1.5 (eBioscience), for CD11c+ cells, clone N418 (Biolegend, San Diego, Calif., USA). PBMCs were obtained from blood drawn from healthy males and females per JHU IRB approved protocols.

1.4.10 Enrichment and Expansion for Small (50-100 nm) Nanoparticle aAPCs

All E+E conditions received the same number of initial splenocytes going into the various isolation conditions. Following cell isolation, the nanoparticle aAPCs were added to cells based on the ratio of 10¹¹ aAPC-bound, peptide-loaded MHC-Ig for every 1×10⁷ splenocytes or for every 10⁶ CD8+ T cells. Similarly, E+Es with KbSIY-only aAPCs or from Pan-T isolate were performed at a ratio of 10¹¹ aAPC-bound peptide-loaded class I MHC-Ig for every 1×10⁶ CD8+ T cells. The aAPC particle and cell mixtures were incubated for 1 h at 4° C. with continual mixing in a PBS buffer with 2 mM EDTA and 0.5% Bovine Serum Albumin (BSA) (Termed Running Buffer). The magnetic particle aAPC: cell mixtures were then separately washed in a Miltenyi MS magnetic column three times. The magnetic column was wet with 0.5 mL of PBS, then the particles/cells were added to the column and washed using two separate washes of B′ Media and third wash using B′ Media with 1% TCGF. The cells were counted using a hemocytometer and plated in a 96 U-bottomed plate in 160 μL per well of B′ Media with 1% TCGF at a concentration of 1×10⁶ splenocytes/mL or 2.5×10⁵ CD8+ T cells/mL. The aAPC:cell mixtures were cultured in a humidified 5% CO₂ 37° C. incubator for 3 days. On day 3, the cells were fed with 80 μL per well of B′ Media with 2% TCGF and placed back into the incubator until day 7. On day 7, the stimulated cells were harvested into a 5-mL round bottom tube for counting and analyzed for antigen specificity by flow cytometry.

1.4.11 96-Well Plate-Based Enrichment and Expansion for 300-Nm aAPCs

A similar protocol to the E+E for small aAPCs, as described above, was followed, with an alternate washing process. Following the 1 h incubation at 4° C., the aAPC:cell mixtures were added to a 96 U-bottomed well plate and placed on a magnet for 5 min. Two different kinds of magnets were used for the experiments the “Ring” magnet—“MAGNUM™ EX Adaptive Magnet Plate” (Alpaqua, Beverly, Mass.)—and the “Bottom” magnet—“EasyPlate Easy Sep Magnet” (STEM-cell, Vancouver, Canada). The buffer was carefully removed from the wells with an angled multichannel pipette to not disrupt the magnetic pellet on the bottom or in the ring. The plate was removed from the magnet, and the pellet was resuspended in 200 μL of B′ Media. The plate was placed on the magnet for 2 min. The supplemented media was then carefully removed from the wells. The plate was removed from the magnet, and the pellet was resuspended in 200 μL of B′ Media with 1% TCGF. Then the plate was placed on the magnet for 2 min. The plate was removed from the magnet, and the pellet was resuspended in 160 μL of B′ Media with 1% TCGF. The plate was placed in a humidified 5% CO₂ 37° C. incubator for 3 days. On day 3, the cells were fed with 80 μL per well of B′ Media with 2% TCGF and placed back into the incubator until day 7. On day 7, the stimulated cells were harvested into a 5-mL round bottom tube for counting and analyzed with antigen-specific staining for flow cytometry. For batched versus individual E+E comparisons, the splenocytes were divided into 8 equal portions in sterile FACS tubes. The respective four types of antigen-specific aAPCs were added to two different FACS tubes. To form the batched condition, four of the individual conditions were combined into one tube and then processed together from then on.

In the case of human T cell expansion, 10% AB serum was used instead of 10% fetal bovine serum. On day 3 of culture, cells were fed with half the volume of the initial T cell culture media with twice the concentration of T cell growth factor cocktail. On day 7 cells were harvested counted and re-plated at a density of 50,000 cells per well with an additional dose of aAPCs, while a subset was taken for antigen-specific staining. On day 10 of culture, cells were fed with half the volume of the initial T cell culture media with twice the concentration of T cell growth factor cocktail. Cells were harvested on day 14, counted, and stained for antigen-specificity.

### 1.4.12 Antigen Specific Staining

On day 7 of culture, the number of cells were counted using a hemocytometer. After counting, less than 500,000 cells were collected and placed into two 5-mL round bottom tubes for antigen-specific staining. One tube was used for the cognate peptide-MHC stain, and the other tube was used for the non-cognate stain to determine background staining. To the two conditions, 1 μg of cognate or non-cognate biotinylated MHC-Ig in 100 μL of PBS with 0.05% sodium azide and 2% FBS (FWB) for 1 h at 4° C. The excess biotinylated MHC-Ig with PBS was washed through centrifugation. The samples were then stained with a 1:350 ratio of PE-labeled streptavidin, with 1:100 APC-conjugated rat anti-mouse CD8a, clone 53-6.7 (Biolegend, San Diego, Calif., USA), and with 1:1000 ratio of LIVE/DEAD® Fixable Green Dead Cell Stain (ThermoFisher) for 15 min at 4° C. Excess secondary and live/dead stain were washed by centrifugation and resuspended with 150 μL of PBS buffer with FWB to read on a BD FACSCalibur flow cytometer. To determine the percent of antigen-specific cells, the following gates were used in the respective order: live+, lymphocyte+(forward scatter by side scatter), CD8+, and Dimer+. The Dimer+gate was determined by comparing non-cognate to the cognate stain. To determine the percentage of antigen-specific cells, the percentage of Dimer+ of the cognate MHC-Ig stain was subtracted from the non-cognate MHC-Ig stain. To obtain the number of antigen-specific cells, this number was multiplied by the percentage of CD8+ T cells and the number of cells counted.

Detection of antigen-specific human cells was done similarly, except instead of staining with biotinylated dimer, the antigen-specific cells were stained with purchased PE-labeled tetramer (MBL International, Woburn, Mass.) for 30 min at room temperature, then washed and stained with APC-conjugated anti-human CD8a, clone SK-1 (Biolegend), and 1:1000 of LIVE/DEAD® Fixable Green Dead Cell Stain for 15 min at 4° C.

### 1.4.13 Fluorescent Magnetic Bead Antigen-Specific Staining

On day 7 of culture, the number of cells were counted using a hemocytometer. After counting, less than 500,000 cells were collected and placed into two 5-mL round bottom tubes for antigen-specific staining. To either tube, pre-loaded MHC-Ig fluorescent beads or adaptive detection beads+/−peptides were added at the indicated amounts and allowed to bind for 45 min at 4° C. Then a solution of 1:100 APC-conjugated rat anti-mouse CD8a, clone 53-6.7 (Biolegend, San Diego, Calif., USA) (for mouse stains) and with 1:1000 ratio of LIVE/DEAD® Fixable Red Dead Cell Stain (ThermoFisher) was added to samples to stain for an additional 15 min at 4° C. Cells were washed and read on a BD FACSCalibur and antigen-specificity was determined similar to biotinylated dimer-MHC staining, while unloaded adaptive detection beads were used as the background staining.

Particle staining of human cells was done using a similar protocol except an APC-conjugated mouse anti-human CD8a, clone SK-1 (Biolegend), was substituted for the rat anti-mouse CD8a stain.

### 1.4.14 In Vivo Peptide Vaccination

Naïve 8-week-old female mice were injected subcutaneously with a mixture of 100 μg SIY peptide and poly I:C diluted into 200 μL PBS on their left rear flanks, and 100 μg VDW peptide and poly I:C diluted into 200 μL PBS on their right rear flanks on both Day 0 and Day 7. On Day 15, mouse spleens and lymph nodes were harvested for dimer and particle staining, following similar protocols described above.

### 1.4.15 Splenocyte Immune Cell Flow Cytometry Panel

Less than 500,000 cells were collected and stained with a 1:100 PBS solution of Pe/Cy7-conjugated rat anti-mouse CD19, clone 6D5 (Biolegend), APC-conjugated rat anti-mouse NK-1.1, clone PK136 (BD Pharmingen), APC/Cy7-conjugated rat anti-mouse CD8a, clone number 53-6.7 (Biolegend), PE-conjugated rat anti-mouse CD4, clone H129.19 (Biolegend), PerCp-conjugated rat anti-mouse CD11c, clone N418 (Biolegend), AmCyan-conjugated F4/80, clone 605 (Biolegend), and 1:1000 of LIVE/DEAD® Fixable Green Dead Cell Stain (ThermoFisher) for 15 min at 4° C. Cells were then washed with FACS wash buffer to be read on BD LSRII flow cytometer and analyzed using FlowJo to measure the population of B cells (CD19+), NK cells (NK1.1+), CD4+ T cells, dendritic cells (CD11c+), and macrophages (F4/80+).

### 1.4.16 T Cell Proliferation Assay

CD8+ T cells were isolated as previous described and resuspended in 1 mL T cell culture media. Cells were mixed with 1 μL CellTrace™ carboxyfluorescein succinimidyl ester (CFSE) dye (ThermoFisher) in 1 mL of T cell culture media per 3 million cells and incubated at 37° C. for 20 min. CFSE stained cells were washed with 50 mL of T cell culture media to remove unstained dye and plated. On day 3 of culture, cells were harvested and stained with a 1:100 PBS solution of APC-conjugated rat anti-mouse CD8a, clone 53-6.7 (Biolegend) for 15 min at 4° C. The CFSE fluorescence intensity was measured using BD FACSCalibur flow cytometer. Cell proliferation was analyzed using FlowJo with diluted CFSE fluorescence peaks signifying population after each round of cell division. A subset of the cells was allowed to expand for 7 days and viable cells were counted with a hemocytometer to determine fold expansion.

### 1.4.17 T Cell Phenotype Assay

On day 7 of culture, the numbers of cells were counted using hemocytometer. After counting, less than 500,000 cells were collected and stained with a 1:100 PBS solution of APC-conjugated rat anti-mouse CD8a, clone 53-6.7 (Biolegend), PE-conjugated rat anti-mouse CD62L, clone MEL-14 (BD Biosciences), PerCP-conjugated rat anti-mouse CD44, clone IM7 (Biolegend), and 1:1000 of LIVE/DEAD® Fixable Green Dead Cell Stain (ThermoFisher) for 15 min at 4° C. Cells were then washed with FACS wash buffer to be read on BD FACSCalibur flow cytometer and analyzed using FlowJo to measure the population of naïve T cells (CD62L+CD44−), effector T cells (CD62L-CD44+), and memory T cells (CD62L+CD44+).

### 1.4.18 T Cell Cytokine Functionality Assay

On day 7 of culture, approximately 500,000 CD8+ T cells were isolated from each condition and separated into cognate or noncognate groups. Cells were stained with 1 μg of either cognate or non-cognate biotinylated pMHC-Ig dimer for 1 h at 4° C. After washing, samples were stained with a 1:350 ratio of PE-labeled streptavidin (BD Pharmingen, San Diego, Calif., USA). Then 10 μL solution of 1:50 FITC anti-CD107a, 1:350 BD GolgiStop Protein Transport Inhibitor (BD Biosciences), and 1:350 BD GolgiPlug Protein Transport Inhibitor (BD Biosciences) in PBS was added to the samples and incubated with 100 μL of complete media for 37° C. for 6 h. Cells were then washed and stained with 1:100 PBS solution of PerCP-conjugated anti-mouse CD8a, clone 53-6.7 (Biolegend) and 1:1000 of LIVE/DEAD® AmCyan Fixable Aqua Dead Cell Stain (ThermoFisher) at 4° C. for 30 min. Cells were then fixed and permeabilized with 100 μL BD Cytofix/Cytoperm Fixation and Permeabilization Solution (BD Biosciences) overnight. Cells were then washed with 1×BD PERM/Wash buffer with 2% BSA and stained with 1:100 solution of APC-conjugated rat anti-mouse IFN-γ, clone XMG1.2 (BD Pharmingen) and PE-Cy7-conjugated rat anti-mouse TNFα, clone MP6-XT22 (Biolegend) in PERM/Wash buffer with 2% BSA at 4° C. for 1 h. Stained cells were read on BD LSR II flow cytometer.

### 1.4.19 IFN-γ Release Assays

On day 7 of culture, 25,000 2C CD8+ T cells were re-stimulated with 300-nm pre-loaded vs. Adaptive aAPCs pre- vs. post-loaded KbSIY/anti-CD28 particles for 18 h at 37° C., and then the supernatants were collected. IFN-γ was measured by ELISA using the ebioscience murine IFN-γ Ready-SET-Go! Kit (San Diego, Calif., USA).

### 1.4.20 Peptide Stabilization Assays

RMA-S cells were left at 25° overnight and pulsed for 2 h with 1 μg peptide and put at 37° for 2 h to degrade unstable MHC molecules. Cells were then stained with anti-Kb clone M1/42 and analyzed by flow cytometry for MHC expression.

### 1.4.21 Doped Enrichment Experiments

PMEL CD8+ T cells were obtained by using a mouse CD8+ T cell negative isolation kit from Miltenyi Biotech and following the manufacturer's instructions. PMEL transgenic mice have CD8+ T cells with the same T cell receptor that recognizes the mouse MHC db loaded with the gp100 peptide. The PMEL CD8+ T cells were counted with a hemocytometer and added at a 1:1000 ratio to wildtype B6 CD8+ T cells and mixed thoroughly in running buffer. Particle aAPCs were added to this mixture at the indicated amounts per 1×10⁶ total CD8+ T cells and allowed to bind at 4° C. for 1 h. The particle cell-mixture was then washed magnetically as previously described within the “Enrichment and Expansion” experiments. All particle-cell mixtures counted via a hemocytometer and stained with the APC-conjugated rat anti-mouse CD8a, clone 53-6.7, (Biolegend) for 15 min at 4° C., washed and read on a BD FACSCalibur.

Fold enrichment was determined by dividing the percent of PMEL positive cells in the eluted particle-cell mixture by the percent of PMEL positive the native 1:1000 doped mixture. Percent cell recovery was calculated by dividing the number of PMEL positive cells in the eluted particle-cell mixture by the number of PMEL positive in the native 1:1000 doped mixture. The PMEL cell counts were calculated by multiplying the number of cells in each mixture by the measured percentages from flow cytometry.

### 1.4.22 Particle and Bead Binding

Particle aAPCs were allowed to bind with cognate transgenic CD8+ T cells at 4° C. for 1 h at various ratios of particle aAPCs to T cells. This mixture was washed and stained with a 1:350 ratio of PE labeled rat-anti-mouse IgG for 15 min at 4° C. PE labeled polyclonal goat-anti-mouse IgG1 (ThermoFisher) recognizes the mouse IgG of the dimeric Kb-Ig on the particles to discriminate the quantitate particles on the surface. Excess antibody was washed away and then stained with a 1:100 PBS solution of APC-conjugated rat anti-mouse CD8a, clone 53-6.7 (Biolegend). Cells were washed and read on a BD FACSCalibur to determine the percent of cells bound with respect to the non-particle bound and non-cognate CD8+ T cells of background staining. Fluorescent magnetic detection bead binding and analysis was performed similarly, except that there is no need for secondary antibody staining since the particles themselves are fluorescent.

### Example 2

### Adaptive aAPCs Pulsed with Tumor-Derived Peptides

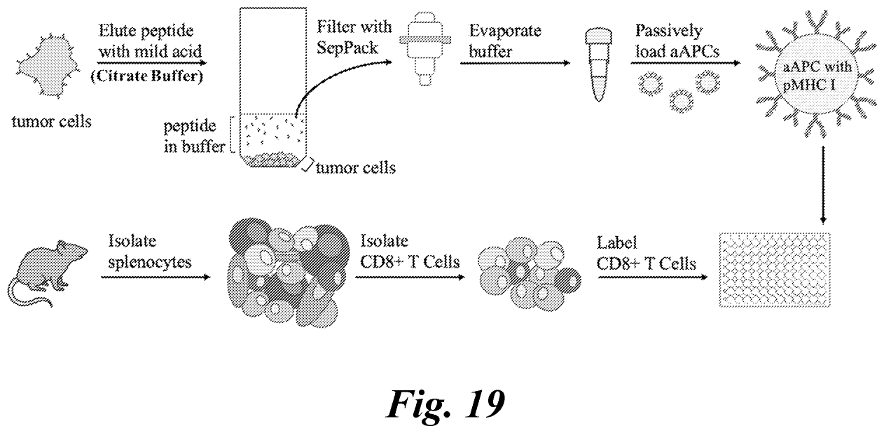

To apply the adaptive aAPC as a potential therapeutic, its utility for presenting tumor-derived peptides was tested. Such an approach would enable an inexpensive off-the-shelf approach for targeting patient-specific tumor epitopes, while bypassing the complex processes involved in identifying targetable candidates. The approach adopted herein combined an established protocol for eluting peptides from MHC I molecules on the surface of tumor cells, Storkus et al., J. Immunother. (1993), with the presently disclosed approach for passively loading adaptive aAPCs. This approach involves incubating tumor cells with a mildly acidic citrate-based buffer (pH 3.3), which destabilizes (32 microglobulin on MHC I molecules, thus releasing presented peptide into the supernatant. Storkus et al., J. Immunother. (1993). This peptide can be isolated from the cell supernatant through solid-phase chromatography and then dried with a vacuum centrifuge. Finally, the peptide can be pulsed onto the presently disclosed adaptive aAPCs and then used to activate tumor-specific T cells (FIG. 19).

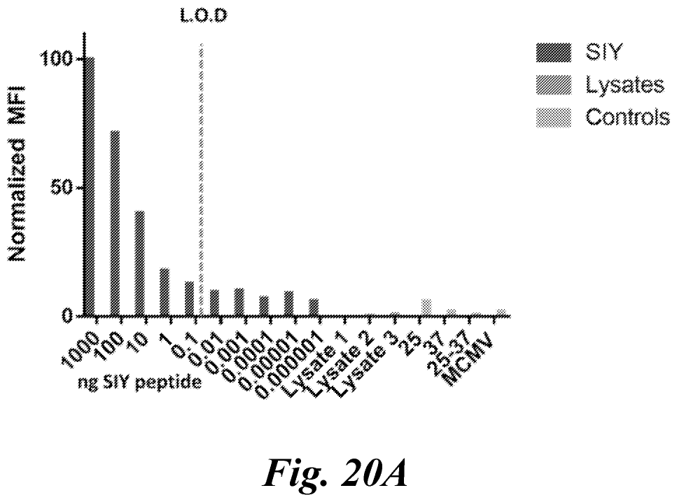

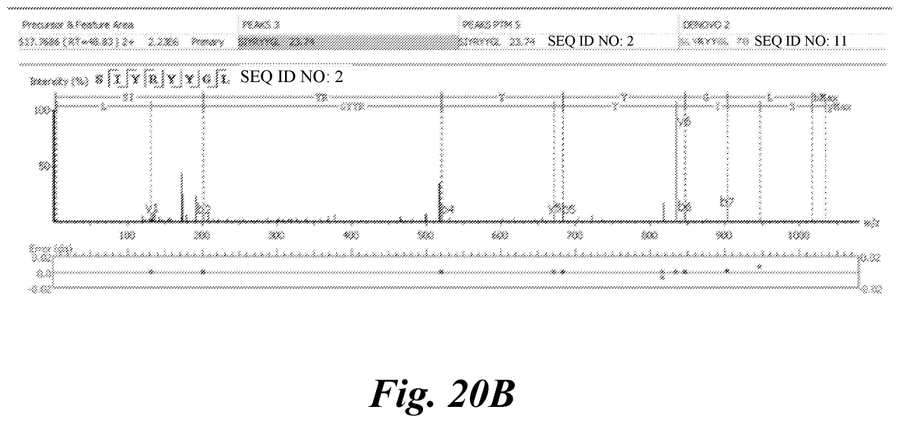

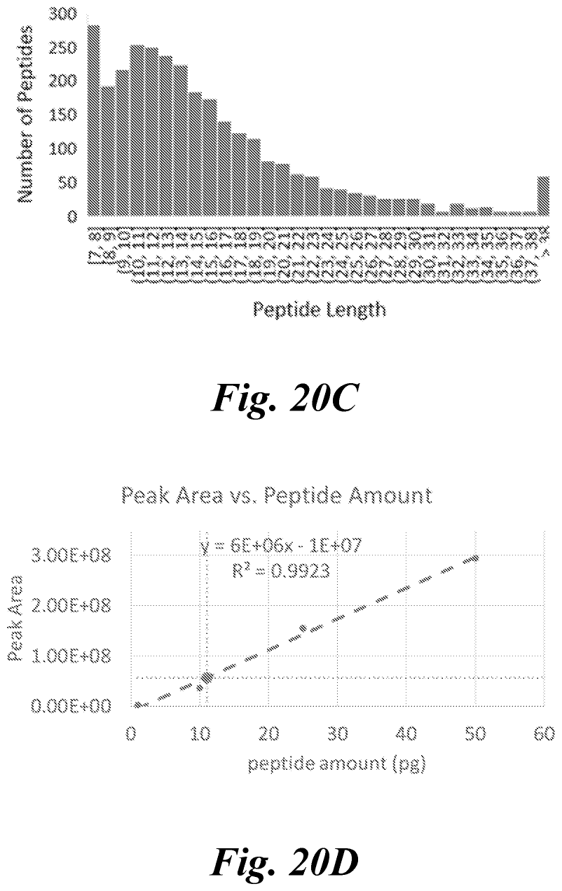

This approach was followed to elute SIY peptide from the surface of 3×10⁴ B16-SIY tumor cells. While the peptide was not able to be detected by the RMA-S assay (FIG. 20A), it could be detected by mass spectrometry (FIG. 20B). That said, it was one of over 3000 spectra detected, suggesting a heterogenous mixture of peptide between 8 and 14 amino acids in length was recovered (FIG. 20C). Through measuring the peak area of a titration of purified SIY peptide on a mass spectrometer, it was determined that half of the eluate contained approximately 11 picograms of SIY peptide (FIG. 20D), indicating that only 0.00002% (w/w) of the 100 micrograms of peptide from 3×10⁴ tumor cells were the SIY peptide.

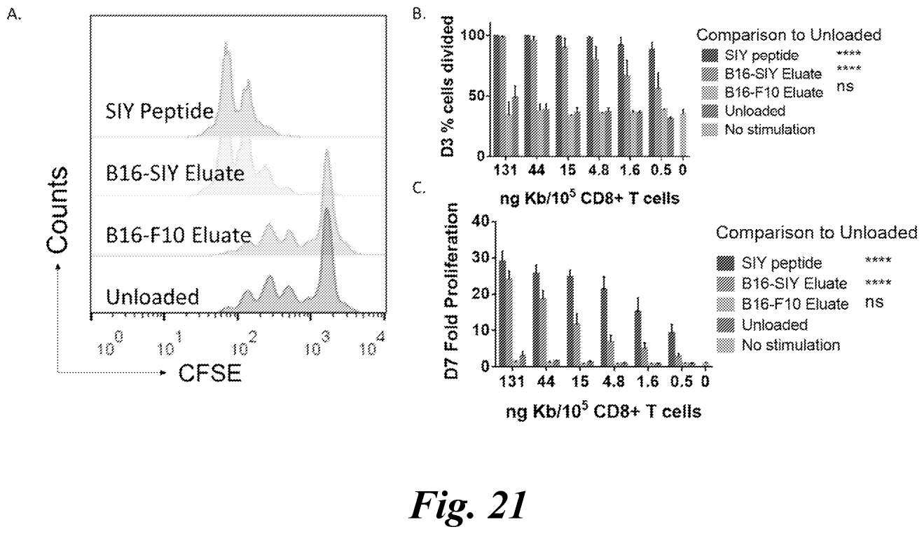

Nevertheless, after being pulsed onto adaptive aAPCs, this heterogenous mixture was able to robustly activate SIY-specific transgenic 2C cells both in terms of CFSE dilutions on Day 3 (FIG. 21A-FIG. 21B) and Fold Proliferation by Day 7 (FIG. 21C) in a manner equivalent to one microgram of pure SIY peptide.

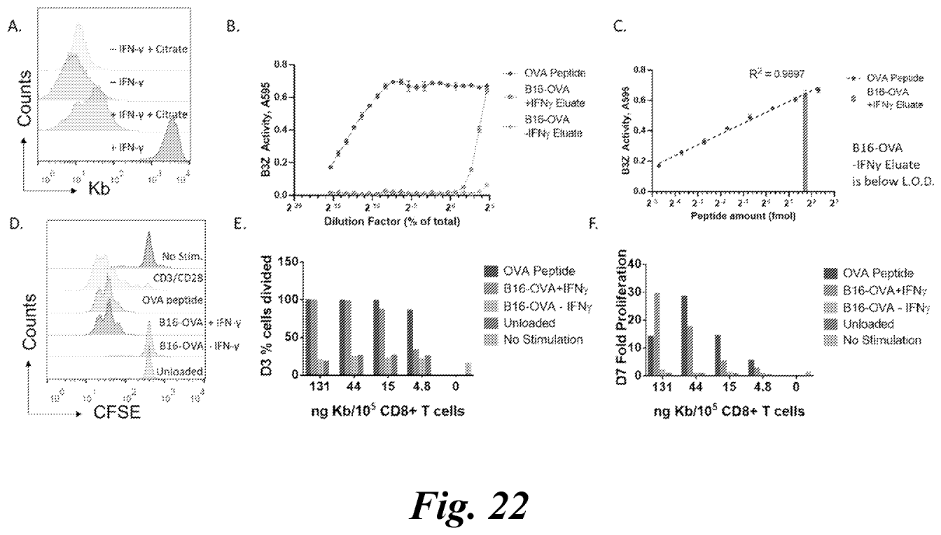

Whether this same approach can be expanded to other tumor models was then examined by applying it to B16-OVA. Initial results were inconclusive, so an attempt to increase the amount of recoverable peptide was made by pre-incubating tumor cells overnight with IFN-γ to upregulate MHC I on their surface. Indeed, IFNγ pre-incubation drastically increased MHC I expression on tumor cells (FIG. 22A) and in-turn increased the amount of recovered peptide, compared to untreated B16-OVA cells (FIG. 22B-FIG. 22C). The calculated yield was approximately 25 μg of peptide per 1×10⁴ tumor cells, still roughly 0.00002% (w/w) of the eluted 100 micrograms of peptide. This small amount of cognate peptide in a heterogenous mixture of irrelevant peptide was potent enough to lead to robust proliferation of OVA-specific CD8+OT-I cells both in terms of CFSE dilutions on day 3 (FIG. 22D—FIG. 22E) and Fold Proliferation by Day 7 (FIG. 22F). In contrast, untreated tumor cells (B16-OVA-IFNγ) did not lead to significant activation or expansion of cognate cells.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Hickey, J. W.; Kosmides, A. K.; Schneck, J. P. Chapter Six—Engineering Platforms for T Cell Modulation. In Biology of T Cells—Part A; Galluzzi, L., Rudqvist, N.-P. B. T.-I. R. of C. and M. B., Eds.; Academic Press, 2018; Vol. 341, pp 277-362.
Singha, S.; Shao, K.; Yang, Y.; Clemente-Casares, X.; Solé, P.; Clemente, A.; Blanco, J.; Dai, Q.; Song, F.; Liu, S. W.; Yamanouchi, J.; Umeshappa, C. S.; Nanjundappa, R. H.; Detampel, P.; Amrein, M.; Fandos, C.; Tanguay, R.; Newbigging, S.; Serra, P.; Khadra, A.; Chan, W. C. W.; Santamaria, P. Peptide-MEIC-Based Nanomedicines for Autoimmunity Function as T-Cell Receptor Microclustering Devices. Nat. Nanotechnol. 2017, 12 (7), 701-710.
Tsai, S.; Shameli, A.; Yamanouchi, J.; Clemente-Casares, X.; Wang, J.; Serra, P.; Yang, Y.; Medarova, Z.; Moore, A.; Santamaria, P. Reversal of Autoimmunity by Boosting Memory-like Autoregulatory T Cells. Immunity 2010, 32 (4), 568-580.
Clemente-Casares, X.; Blanco, J.; Ambalavanan, P.; Yamanouchi, J.; Singha, S.; Fandos, C.; Tsai, S.; Wang, J.; Garabatos, N.; Izquierdo, C.; Agrawal, S.; Keough, M. B.; Yong, V. W.; James, E.; Moore, A.; Yang, Y.; Stratmann, T.; Serra, P.; Santamaria, P. Expanding Antigen-Specific Regulatory Networks to Treat Autoimmunity. Nature 2016,530 (7591), 434-440.
Riddell, S. R.; Elliott, M.; Lewinsohn, D. A.; Gilbert, M. J.; Wilson, L.; Manley, S. A.; Lupton, S. D.; Overell, R. W.; Reynolds, T. C.; Corey, L. T—Cell Mediated Rejection of Gene—Modified HIV—Specific Cytotoxic T Lymphocytes in HIV—Infected Patients. Nat. Med. 1996, 2 (2), 216.
Walter, E. A.; Greenberg, P. D.; Gilbert, M. J.; Finch, R. J.; Watanabe, K. S.; Thomas, E. D.; Riddell, S. R. Reconstitution of Cellular Immunity against Cytomegalovirus in Recipients of Allogeneic Bone Marrow by Transfer of T-Cell Clones from the Donor. N Engl. J. Med. 1995, 333 (16), 1038-1044.
Fesnak, A. D.; June, C. H.; Levine, B. L. Engineered T Cells: The Promise and Challenges of Cancer Immunotherapy. Nat. Rev. Cancer 2016, 16 (9), 566-581.
Rapoport, A. P.; Stadtmauer, E. A.; Binder-Scholl, G. K.; Goloubeva, 0.; Vogl, D. T.; Lacey, S. F.; Badros, A. Z.; Garfall, A.; Weiss, B.; Finklestein, J. NY-ESO-1—Specific TCR—Engineered T Cells Mediate Sustained Antigen-Specific Antitumor Effects in Myeloma. Nat. Med. 2015, 21 (8), 914.
Davis, M. M.; Bjorkman, P. J. T-Cell Antigen Receptor Genes and T-Cell Recognition. Nature 1988, 334 (6181), 395.
Jenkins, M. K.; Moon, J. J. The Role of Naive T Cell Precursor Frequency and Recruitment in Dictating Immune Response Magnitude. J. Immunol. 2012, 188 (9), 4135-4140.
Rizzuto, G. A.; Merghoub, T.; Hirschhorn-Cymerman, D.; Liu, C.; Lesokhin, A. M.; Sahawneh, D.; Zhong, H.; Panageas, K. S.; Perales, M.-A.; Altan-Bonnet, G. Self-Antigen—Specific CD8+ T Cell Precursor Frequency Determines the Quality of the Antitumor Immune Response. J. Exp. Med. 2009, 206 (4), 849-866.
Newell, E. W.; Davis, M. M. Beyond Model Antigens: High-Dimensional Methods for the Analysis of Antigen-Specific T Cells. Nat. Biotechnol. 2014, 32 (2), 149.
Altman, J. D.; Moss, P. A. H.; Goulder, P. J. R.; Barouch, D. H.; McHeyzer-Williams, M. G.; Bell, J. I.; McMichael, A. J.; Davis, M. M. Phenotypic Analysis of Antigen-Specific T Lymphocytes. Science (80-.). 1996, 274 (5284), 94-96.
Han, Q.; Bradshaw, E. M.; Nilsson, B.; Hafler, D. A.; Love, J. C. Multidimensional Analysis of the Frequencies and Rates of Cytokine Secretion from Single Cells by Quantitative Microengraving. Lab Chip 2010, 10 (11), 1391-1400.
Betts, M. R.; Brenchley, J. M.; Price, D. A.; De Rosa, S. C.; Douek, D. C.; Roederer, M.; Koup, R. A. Sensitive and Viable Identification of Antigen-Specific CD8+ T Cells by a Flow Cytometric Assay for Degranulation. J. Immunol. Methods 2003, 281 (1-2), 65-78.
Frentsch, M.; Arbach, O.; Kirchhoff, D.; Moewes, B.; Worm, M.; Rothe, M.; Scheffold, A.; Thiel, A. Direct Access to CD4+ T Cells Specific for Defined Antigens According to CD154 Expression. Nat. Med. 2005, 11 (10), 1118.
Newell, E. W.; Sigal, N.; Bendall, S. C.; Nolan, G. P.; Davis, M. M. Cytometry by Time-of-Flight Shows Combinatorial Cytokine Expression and Virus-Specific Cell Niches within a Continuum of CD8+ T Cell Phenotypes. Immunity 2012, 36 (1), 142-152.
Perica, K.; Bieler, J. G.; Schütz, C.; Varela, J. C.; Douglass, J.; Skora, A.; Chiu, Y. L.; Oelke, M.; Kinzler, K.; Zhou, S. Enrichment and Expansion with Nanoscale Artificial Antigen Presenting Cells for Adoptive Immunotherapy. ACS Nano 2015, 9 (7), 6861-6871.
Day, C. L.; Seth, N. P.; Lucas, M.; Appel, H.; Gauthier, L.; Lauer, G. M.; Robbins, G. K.; Szczepiorkowski, Z. M.; Casson, D. R.; Chung, R. T.; Bell, S.; Harcourt, G.; Walker, B. D.; Klenerman, P.; Wucherpfennig, K. W. Ex Vivo Analysis of Human Memory CD4 T Cells Specific for Hepatitis C Virus Using MHC Class II Tetramers. J. Clin. Invest. 2003, 112 (6), 831-842. https://doi.org/10.1172/JCI18509.
Rodenko, B.; Toebes, M.; Hadrup, S. R.; Van Esch, W. J. E.; Molenaar, A. M.; Schumacher, T. N. M.; Ovaa, H. Generation of Peptide—MHC Class I Complexes through UV-Mediated Ligand Exchange. Nat. Protoc. 2006, 1 (3), 1120.
Hadrup, S. R.; Bakker, A. H.; Shu, C. J.; Andersen, R. S.; Van Veluw, J.; Hombrink, P.; Castermans, E.; thor Straten, P.; Blank, C.; Haanen, J. B. Parallel Detection of Antigen-Specific T-Cell Responses by Multidimensional Encoding of MHC Multimers. Nat. Methods 2009, 6 (7), 520.
Newell, E. W.; Klein, L. O.; Yu, W.; Davis, M. M. Simultaneous Detection of Many T-Cell Specificities Using Combinatorial Tetramer Staining. Nat. Methods 2009, 6 (7), 497.
Gee, M. H.; Han, A.; Lofgren, S. M.; Beausang, J. F.; Mendoza, J. L.; Birnbaum, M. E.; Bethune, M. T.; Fischer, S.; Yang, X.; Gomez-Eerland, R. Antigen Identification for Orphan T Cell Receptors Expressed on Tumor-Infiltrating Lymphocytes. Cell 2018, 172 (3), 549-563.
Hickey, J. W.; Isser, A. Y.; Vicente, F. P.; Warner, S. B.; Mao, H.-Q.; Schneck, J. P. Efficient Magnetic Enrichment of Antigen-Specific T Cells by Engineering Particle Properties. Biomaterials 2018, 187, 105-116.
Hickey, J. W.; Schneck, J. P. Enrich and Expand Rare Antigen-Specific T Cells with Magnetic Nanoparticles. JoVE (Journal Vis. Exp.) 2018, No. 141, e58640.
Kosmides, A. K.; Necochea, K.; Hickey, J. W.; Schneck, J. P. Separating T Cell Targeting Components onto Magnetically Clustered Nanoparticles Boosts Activation. Nano Lett. 2018, 18 (3).
Inaba, K.; Young, J. W.; Steinman, R. M. Direct Activation of CD8+Cytotoxic T Lymphocytes by Dendritic Cells. J. Exp. Med. 1987, 166 (1), 182-194.
Novy, P.; Quigley, M.; Huang, X.; Yang, Y. CD4 T Cells Are Required for CD8 T Cell Survival during Both Primary and Memory Recall Responses. J. Immunol. 2007, 179 (12), 8243 LP—8251.
Hickey, J. W. Vicente, F. P.; Howard, G. P. Mao, H.-Q.; Schneck, J. P. J. P. Biologically Inspired Design of Nanoparticle Artificial Antigen-Presenting Cells for Immunomodulation. Nano Lett. 2017, 17 (11), 7045-7054.
Yarchoan, M.; Johnson III, B. A.; Lutz, E. R.; Laheru, D. A.; Jaffee, E. M. Targeting Neoantigens to Augment Antitumour Immunity. Nat. Rev. Cancer 2017, 17 (4), 209.
Schumacher, T. N.; Schreiber, R. D. Neoantigens in Cancer Immunotherapy. Science (80-.). 2015, 348 (6230), 69-74.
Abiko, K.; Matsumura, N.; Hamanishi, J.; Horikawa, N.; Murakami, R.; Yamaguchi, K.; Yoshioka, Y.; Baba, T.; Konishi, I.; Mandai, M. IFN-γ from Lymphocytes Induces PD-L1 Expression and Promotes Progression of Ovarian Cancer. Br. J. Cancer 2015, 112 (9), 1501.
Sahin, U.; Derhovanessian, E.; Miller, M.; Kloke, B.-P.; Simon, P.; Lower, M.; Bukur, V.; Tadmor, A. D.; Luxemburger, U.; Schrors, B.; Omokoko, T.; Vormehr, M.; Albrecht, C.; Paruzynski, A.; Kuhn, A. N.; Buck, J.; Heesch, S.; Schreeb, K. H.; Muller, F.; Ortseifer, I.; Vogler, I.; Godehardt, E.; Attig, S.; Rae, R.; Breitkreuz, A.; Tolliver, C.; Suchan, M.; Martic, G.; Hohberger, A.; Sorn, P.; Diekmann, J.; Ciesla, J.; Waksmann, O.; Bruck, A.-K.; Witt, M.; Zillgen, M.; Rothermel, A.; Kasemann, B.; Langer, D.; Bolte, S.; Diken, M.; Kreiter, S.; Nemecek, R.; Gebhardt, C.; Grabbe, S.; Holler, C.; Utikal, J.; Huber, C.; Loquai, C.; Tureci, O. Personalized RNA Mutanome Vaccines Mobilize Poly-Specific Therapeutic Immunity against Cancer. Nature 2017, 547 (7662), 222-226.
Gonzalez, S.; Volkova, N.; Beer, P.; Gerstung, M. Immuno-Oncology from the Perspective of Somatic Evolution. Semin. Cancer Biol. 2018, 52, 75-85.
Sarkizova, S.; Hacohen, N. How T Cells Spot Tumour Cells. Nature 2017, 551 (7681), 444-446.
Topalian, S. L.; Taube, J. M.; Anders, R. A.; Pardoll, D. M. Mechanism-Driven Biomarkers to Guide Immune Checkpoint Blockade in Cancer Therapy. Nat. Rev. Cancer 2016, 16 (5), 275-287.
Castle, J. C.; Kreiter, S.; Diekmann, J.; Löwer, M.; Van De Roemer, N.; De Graaf, J.; Selmi, A.; Diken, M.; Boegel, S.; Paret, C.; Koslowski, M.; Kuhn, A. N.; Britten, C. M.; Huber, C.; Türeci, Ö.; Sahin, U. Exploiting the Mutanome for Tumor Vaccination. Cancer Res. 2012, 72 (5), 1081-1091.
Storkus, W. J., Zeh, H. J. 3rd, Salter, R. D. & Lotze, M. T. Identification of T-cell epitopes: rapid isolation of class I-presented peptides from viable cells by mild acid elution. J. of Immunother. with Emphasis on Tumor Immunology 14, 94-103 (1993).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing one more adaptive artificial antigen presenting cells (aAPCs), the method comprising:
(a) conjugating to a surface of a paramagnetic particle a major histocompatibility complex (MHC) or a human leukocyte antigen (HLA) and a costimulatory ligand to form a conjugated paramagnetic particle, wherein:
(i) the magnetic particle has a diameter ranging from about 100 nm to about 5000 nm; and
(ii) the MHC or HLA is not loaded with a peptide prior to conjugating the MHC or HLA to the surface of the paramagnetic particle; and
(b) incubating the conjugated paramagnetic particle with one or more peptides to load the conjugated MHC or HLA on the paramagnetic particle with the one or more peptides to form one or more adaptive aAPCs.

2. The method of claim 1, wherein the major histocompatibility complex (MHC) is selected from the group consisting of an MHC-class I complex and an MHC-class II complex.

3. The method of claim 3, wherein the MHC-class I complex or MHC-class II complex comprises an MHC-Ig dimer.

4. The method of any one of claims 1-3, wherein the costimulatory ligand is selected from the group consisting of an antibody or antigen-binding fragment thereof that specifically binds to CD28, CD80 (B7-1), CD86 (B7-2), B7-H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB.

5. The method of claim 1, wherein the one or more peptides can be the same or different and can be loaded individually or simultaneously from a mixture of 10,000 or more distinct antigens.

6. The method of claim 5, wherein the one or more peptides are eluted and/or isolated from a tumor cell or other cell expressing an antigen of interest.

7. The method of any of claims 1-6, wherein each distinct antigen is loaded onto the conjugated paramagnetic particle in a separate well of a multi-well microtiter plate.

8. The method of any of claim 7, further comprising washing the adaptive aAPCs magnetically.

9. An adaptive aAPC prepared by the method of any one of claims 1-8.

10. An adaptive artificial antigen presenting cell (aAPC) comprising a paramagnetic particle having a major histocompatibility complex (MHC) or a human leukocyte antigen (HLA) and a costimulatory ligand conjugated to a surface thereof, wherein:
(a) the magnetic particle has a diameter ranging from about 100 nm to about 5000 nm; and
(b) is capable of loading one or more antigen peptides through binding with the MHC or HLA prior to contacting T cells.

11. The adaptive aAPC of claim 10, wherein the major histocompatibility complex (MHC) is selected from the group consisting of an MHC-class I complex and an MHC-class II complex.

12. The adaptive aAPC of claim 11, wherein the MHC-class I complex or MHC-class II complex comprises an MHC-Ig dimer.

13. The adaptive aAPC of any one of claims 10-12, wherein the costimulatory ligand is selected from the group consisting of an antibody or antigen-binding fragment thereof that specifically binds to CD28, CD80 (B7-1), CD86 (B7-2), B7-H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB.

14. The adaptive aAPC of any one of claims 10-13, wherein the one or more peptides can be loaded individually or simultaneously from a mixture of 10,000 or more distinct antigens.

15. The adaptive aAPC of claim 14, wherein the one or more peptides are eluted and/or isolated from a tumor cell or other cell expressing an antigen of interest.

16. A method for preparing one or more adaptive detection beads, the method comprising:
(a) conjugating to a surface of a paramagnetic particle a major histocompatibility complex (MHC) or a human leukocyte antigen (HLA) to form a conjugated paramagnetic particle, wherein:
(i) the magnetic particle has a diameter ranging from about 100 nm to about 5000 nm;
(ii) the MHC or HLA is not loaded with a peptide prior to conjugating the MHC or the HLA to the surface of the paramagnetic particle; and
(iii) the magnetic particle is labeled with a reporting moiety; and
(b) incubating the conjugated paramagnetic particle with one or more peptides to load the conjugated MHC or HLA on the paramagnetic particle with the one or more peptides to form one or more adaptive detection beads.

17. The method of claim 16, wherein the major histocompatibility complex (MHC) is selected from the group consisting of an MHC-class I complex and an MHC-class II complex.

18. The method of claim 17, wherein the MHC-class I complex or the MHC-class II complex comprises an MHC-Ig dimer.

19. The method of claim 16, wherein the one or more peptides can be loaded individually or simultaneously from a mixture of 10,000 or more distinct antigens.

20. The method of claim 19, wherein the one or more peptides are eluted and/or isolated from a tumor cell or other cell expressing an antigen of interest.

21. The method of any of claims 16-20, wherein each distinct antigen is loaded onto the conjugated paramagnetic particle in a separate well of a multi-well microtiter plate.

22. The method of claim 21, further comprising washing the adaptive detection bead magnetically.

23. The method of any one of claims 16-22, wherein the reporting moiety comprises a fluorescent agent.

24. An adaptive detection bead prepared by the method of any one of claims 16-23.

25. An adaptive detection bead comprising a paramagnetic particle having a major histocompatibility complex (MHC) or a human leukocyte antigen (HLA) conjugated to a surface thereof, wherein:
(a) the magnetic particle has a diameter ranging from about 100 nm to about 5000 nm;
(b) is capable of loading one or more antigen peptides through binding with the MHC or HLA prior to contacting T cells; and
(c) the magnetic particle is labeled with a reporting moiety.

26. The adaptive detection bead of claim 25, wherein the major histocompatibility complex (MHC) is selected from the group consisting of an MHC-class I complex and an MHC-class II complex.

27. The adaptive detection bead of claim 26, wherein the MHC-class I complex or MHC-class II complex comprises an MHC-Ig dimer.

28. The adaptive detection bead of any one of claims 25-27, wherein the one or more peptides can be loaded individually or simultaneously from a mixture of 10,000 or more distinct antigens.

29. The adaptive detection bead of claim 28, wherein the one or more peptides are eluted and/or isolated from a tumor cell or other cell expressing an antigen of interest.

30. The adaptive detection bead of any one of claims 25-29, wherein the reporting moiety comprises a fluorescent agent.

31. A method for identifying, isolating, or detecting one or more antigen-specific T cells, the method comprising:
(a) contacting a plurality of unpurified immune cells comprising one or more antigen-specific T cells with a plurality of adaptive aAPCs prepared by the method of any one of claims 1-8 and/or a plurality of adaptive detection beads prepared by the method of any one of claims 16-25;
(b) placing a magnetic field in proximity to the plurality of adaptive aAPCs and/or the plurality of adaptive detection beads to separate antigen-specific T cells associated with the plurality of adaptive aAPCs and/or the plurality of adaptive detection beads from cells not associated with the plurality of adaptive aAPCs and/or the plurality of adaptive detection beads;
(c) recovering antigen-specific T cells associated with the plurality of adaptive aAPCs and/or the plurality of adaptive detection beads; and
(d) expanding the recovered antigen-specific T cells in culture for a period of time to provide a composition comprising antigen-specific T cells.

32. The method of claim 31, wherein the plurality of unpurified immune cells comprising one or more antigen-specific T cells are obtained from a sample comprising one or more of a peripheral blood mononuclear cell (PBMC) sample, memory T cells, naive T cells, previously activated T cells, and tumor infiltrating lymphocytes.

33. The method of claim 31 or claim 32, wherein the plurality of unpurified immune cells comprising one or more antigen-specific T cells are obtained from a sample comprising one or more of bone marrow, lymph node tissue, spleen tissue, and a tumor.

34. The method of any one of claims 31-33, wherein the plurality of unpurified immune cells are obtained from a patient or a donor.

35. The method of claim 34, wherein the donor comprises a donor who is HLA-matched to an adoptive transfer recipient.

36. The method of claim 35, wherein the plurality of unpurified immune cells are obtained from a patient and the patient has one or more diseases, disorders, or conditions selected from the group consisting of a cancer, an infectious disease, and an autoimmune disease.

37. The method of any one of claims 31-36, wherein the one or more antigen-specific T cells are selected from the group consisting of cytotoxic T lymphocytes, helper T cells, and regulatory T cells.

38. The method of claim 37, wherein the one or more antigen-specific T cells are selected from the group consisting of CD8+ cytotoxic T lymphocytes, CD4+ helper T cells, and combinations thereof.

39. The method of any one of claims 31-38, wherein the magnetic field comprises a magnetic field associated with a permanent magnet.

40. The method of any one of claims 31-39, wherein the magnetic field comprises a magnetic field associated with a neodymium magnet.

41. The method of any one of claims 31-40, wherein the expanding of the recovered cells in culture for a period of time is performed on a multi-well microtiter plate.

42. The method of claim 41, wherein the multi-well microtiter plate comprises a 96-well microtiter plate.

43. The method of any one of claims 31-42, wherein a purity of the expanded recovered antigen-specific T cells is improved relative to a method in which the antigen-specific T cells are isolated from the plurality of unpurified immune cells prior to contacting the plurality of unpurified immune cells with the plurality of paramagnetic nanoparticles.

44. The method of any one of claims 31-42, wherein a percent of antigen-specific T cells is increased relative to a method in which the antigen-specific T cells are isolated from the plurality of unpurified immune cells prior to contacting the plurality of unpurified immune cells with the plurality of paramagnetic nanoparticles.

45. The method of any one of claims 29-42, wherein a number of antigen-specific T cells is increased relative to a method in which the antigen-specific T cells are isolated from the plurality of unpurified immune cells prior to contacting the plurality of unpurified immune cells with the plurality of paramagnetic nanoparticles.

46. A method for treating a disease, disorder, or condition, the method comprising administering to a subject in need of treatment thereof a composition comprising one or more antigen-specific T cells prepared by the method of any one of claims 31-45.

47. The method of claim 46, wherein the disease, disorder, or condition is selected from the group consisting of a cancer, an infectious disease, and an autoimmune disease.

48. The method of claim 47, wherein the disease, disorder, or condition is a cancer and the one or more antigen-specific T cells comprise cytotoxic T cells specific for one or more tumor-associated peptide antigens to the subject in need of treatment thereof.

50. The method of claim 48, wherein the cancer comprises a solid tumor or a hematological malignancy.

51. The method of claim 49, wherein the cancer is selected from the group consisting of a melanoma, colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, nonsmall cell lung carcinoma, small-cell lung cancer, mesothelioma, transitional and squamous cell urinary carcinoma, brain cancer, a neuroblastoma, and a glioma.


SEQUENCE LISTING (11 sequences)

SEQ ID NO: 1 - protein, 9 aa, Artificial sequence
  Synthetic peptide
KVPRNQDWL

SEQ ID NO: 2 - protein, 8 aa, Artificial sequence
  Synthetic peptide
SIYRYYGL

SEQ ID NO: 3 - protein, 8 aa, Artificial sequence
  Synthetic peptide
SIYRYYGL

SEQ ID NO: 4 - protein, 8 aa, Artificial sequence
  Synthetic peptide
SIINFEKL

SEQ ID NO: 5 - protein, 9 aa, Artificial sequence
  Synthetic peptide
SVYDFFVWL

SEQ ID NO: 6 - protein, 10 aa, Artificial sequence
  Synthetic peptide
VDWENVSPEL

SEQ ID NO: 7 - protein, 9 aa, Artificial sequence
  Synthetic peptide
YPHFMPTNL

SEQ ID NO: 8 - protein, 9 aa, Artificial sequence
  Synthetic peptide
NLVPMVATV

SEQ ID NO: 9 - protein, 9 aa, Artificial sequence
  Synthetic peptide
GILGFVFTL

SEQ ID NO: 10 - protein, 10 aa, Artificial sequence
  Synthetic peptide
ELAGIGILTV

SEQ ID NO: 11 - protein, 8 aa, Artificial sequence
  Synthetic peptide
SLYRYYGL